(19) United States
(12) Patent Application Publication
(10) Pub. No.: US 2010/0247547 A1
(43) Pub. Date: Sep. 30, 2010

(54) MODULATION OF THE TH-17 CELL MEDIATED IMMUNE RESPONSES

(76) Inventors: Chen Dong, Bellaire, TX (US); Roza Nurieva, Manvel, TX (US)

Correspondence Address: Nielsen IP Law LLC, 1177 West Loop South, Suite 1600, Houston, TX, 77027, US

(21) Appl. No.: 12/602,114

(22) PCT Filed: May 29, 2008
(86) PCT No.: PCT/US08/65114

§ 371 Date: May 19, 2010

Related U.S. Application Data
(60) Provisional application No. 60/940,600, filed on May 29, 2007.

(51) Int. Cl. A61K 39/395; C12N 5/0783; A61P 37/04

(52) U.S. Cl. 424/158.1; 435/377

(57) ABSTRACT

Methods of treating diseases or conditions associated with T_{H} 17 cell mediated immune response are provided where a subject is administered an effective amount of an agent that modulates the IL-21 signaling pathway so as to inhibit or induce the differentiation of T_{H}17 cells and/or the expression of IL-17, IL17-F, IL-22, and IL-21.

### CROSS-REFERENCE TO RELATED APPLICATIONS

This application claims priority to U.S. Pat. App. Ser. No. 60/940,600 filed May 29, 2007. This application is incorporated by reference herein it its entirety.

### STATEMENT REGARDING FEDERALLY SPONSORED RESEARCH OR DEVELOPMENT

This disclosure was developed at least in part using funding from the National Institutes of Health, Grant No. R01-AR050772. The U.S. government may have certain rights in this invention.

### FIELD OF THE INVENTION

Methods of modulating T_{H}17 mediated immune responses and associated diseases and disorders are provided.

### THE NAMES OF THE PARTIES TO A JOINT RESEARCH AGREEMENT

None.

### REFERENCE TO SEQUENCE LISTING

None.

### BACKGROUND OF THE INVENTION

After activation, CD4+ helper T (T_{H}) cells differentiate into distinct effector subsets that are characterized by their unique cytokine expression and immunoregulatory function. Dong, C., et al., Cell Fate Decision: T-Helper 1 and 2 Subsets in Immune Responses, 2000, Arthritis Res 2, 179-188; Glimcher, L., et al., Lineage Commitment in the Immune System: The T Helper Lymphocyte Grows Up, 2000, Genes Dev. 14, 1693-1711. During this differentiation, T_{H}1 and T_{H}2 cells produce IFNγ and IL-4, respectively, as autocrine factors necessary for selective lineage commitment.

Another T_{H} subset, T_{H}IL-17, has been recently identified as a distinct T_{H} lineage. TH_{IL-17} cells mediate tissue inflammation. Dong, C., Diversification of T-Helper-Cell Lineages: Finding the Family Root of IL-17-Producing Cells, 2006, Nat Rev Immunol 6, 329-34; Weaver, C. T., et al., TH17: An Effector CD4 T Cell Lineage With Regulatory T Cell Ties, 2006, Immunity 24, 677-88. TH_{IL-17} cells are a subset of CD4+ helper T (T_{H}) cells. This cell type is also referred to sometimes as T_{H}17 cells, TH-17 cells, TH17 cells, inflammatory TH (THi) cells, IL-17-producing CD4⁺ T cells, IL-17-producing cells, T_{H}17, TH-17, and TH17.

T_{H}17 cell differentiation is initiated by TGFβ and IL-6 and is known to be further advanced by IL-23. STAT3 and retinioic acid receptor-related orphan receptor (“RORγ”), which is encoded by Rorc, mediate this specific lineage. Bettelli, E., et al., Reciprocal Developmental Pathways For the Generation of Pathogenic Effector TH17 and Regulatory T Cells, 2006, Nature 441, 235-8; Mangan, P. R., et al., Transforming Growth Factor-Beta Induces Development of the T(H)17 Lineage, 2006, Nature 441, 231-4; Veldhoen, M., et al., TGFbeta in the Context of an Inflammatory Cytokine Milieu Supports De Novo Differentiation of IL-17-Producing T Cells, 2006, Immunity 24, 179-89; Yang, X. O., et al., STAT3 Regulates Cytokine-Mediated Generation of Inflammatory Helper T Cells, 2007, J Biol Chem 282, 9358-9363; Chen, Z., et al., Selective Regulatory Function of Socs3 in the Formation of IL-17-Secreting T Cells, 2006, Proc Natl Acad Sci USA 103, 8137-42; Ivanov, I I, et al., The Orphan Nuclear Receptor RORgammat Directs the Differentiation Program of Proinflammatory IL-17+ T Helper Cells, 2006, Cell 126, 1121-33.

T_{H}17 cells express IL-17, IL-17F and IL-22, all of which regulate inflammatory responses by tissue cells yet have no function in TH17 differentiation. Chung, Y., et al., Expression and Regulation of IL-22 in the IL-17-Producing CD4+ T Lymphocytes, 2006, Cell Res 16, 902-7; Langrish, C. L., et al., IL-23 Drives a Pathogenic T Cell Population That Includes Autoimmune Inflammation, 2005, J Exp Med 201, 233-40; Liang, S. C., et al., Interleukin (IL)-22 and IL-17 Are Coexpressed by Th17 Cells and Cooperatively Enhance Expression of Antimicrobial Peptides, 2006, J Exp Med 203, 2271-9; Zheng, Y, et al., Interleukin-22, a TH17 Cytokine, Mediates IL-23-Induced Dermal Inflammation and Acanthosis, 2007, Nature 445, 648-651.

A need exists, therefore, to understand what causes differentiation of T_{H}17 cells so as to further understand immune responses, and treat associated diseases, disorders and conditions.

### BRIEF SUMMARY OF THE INVENTION

Methods of modulating T_{H}17 cell mediated immune response by inhibiting or inducing IL-21 signaling are provided. These methods also provide modulating the expression and/or activity of IL-21 to mediate T_{H}17 cell immune response including the differentiation of T_{H}17 cells.

Methods of treating an inflammatory disease or condition in a subject are further provided. These methods are useful for treating and/or preventing inflammatory diseases. The methods provided herein may also be used in diseases or conditions associated with inflammation. These methods of treatment include administering an agent to modulate IL-21 signaling, expression or activity in an amount effective to modulate the differentiation of T_{H}17 cells. Hence, a useful agent may modulate the production, growth or activity of the T_{H}17 cell. This agent may also modulate the expression of IL-17, IL17-F, or IL-22, and/or be used in combination with other agents. Further provided are methods of modulating the function and/or generation of T_{H}17 cells by use of an agent such as an IL-21 antagonist and/or an IL-21 agonist in an amount effective to inhibit or induce the differentiation of T_{H}17 cells and/or modulate the expression of IL-17, IL17-F, IL-22, and IL-21 by T_{H}17 cells.

### BRIEF DESCRIPTION OF THE SEVERAL VIEWS OF THE DRAWINGS

FIG. 1A depicts expression of the indicated mRNAs by various T_{H} cell lineages. OT-II T cells differentiated under various conditions were re-stimulated with anti-CD3 for 4 hours for real-time reverse-transcriptase-mediated polymerase chain reaction (“RT-PCR”) analysis.

FIG. 1B depicts expression of IL-21 when naive B6 T cells were activated with anti-CD3, anti-CD28 and IL-2 with indicated cytokines for 2 days (left) or 5 days (right). After re-stimulation with anti-CD3, cytokine mRNA expression was analyzed by RT-PCR.

FIG. 1C depicts expression of IL-21 when naive T cells from wild-type (open columns) and STAT3-deficient (filled columns) mice were activated as indicated. T cells were re-stimulated for IL-21 mRNA expression. The RT-PCR data shown were normalized to b-actin levels, and expression in neutral conditions was set as 1.0. The results shown are representative of at least two independent experiments.

FIG. 1D depicts expression of IL-21 when naive T cells from wild-type (open columns) and ROR-γ-deficient (filled columns) mice were activated as indicated. T cells were re-stimulated for IL-21 mRNA expression. The RT-PCR data shown were normalized to b-actin levels, and expression in neutral conditions was set as 1.0. The results shown are representative of at least two independent experiments.

FIG. 2A shows IFN-γ and IL-17 production or Foxp3 expression after naïve B6 T cells were activated as indicated and subsequently analyzed by intracellular staining.

FIG. 2B depicts mRNA expression of indicated genes after naïve B6 T cells were activated as indicated and subsequently analyzed by analyzed by real-time RT-PCR.

FIG. 2C depicts the results when naive T cells from STAT3 KO mice (filled columns) and their appropriate controls (open columns) were activated and analyzed as above. Numbers in dot-plot quadrants show percentages. The data represent at least two independent experiments with consistent results. WT, wild type.

FIG. 2D depicts the results when naïve T cells from ROR-γ KO mice (filled columns) and their appropriate controls (open columns) were activated and analyzed as above. Numbers in dot-plot quadrants show percentages. The data represent at least two independent experiments with consistent results. WT, wild type.

FIG. 3A shows IFN-γ and IL-17 production after naive T_{H} cells from IL-21 KO mice and their littermate control mice were activated with the indicated cytokines and neutralizing antibodies. Five days later, cells were assessed for IFN-γ and IL-17 production by using intracellular staining. Numbers in quadrants are percentages.

FIG. 3B shows Foxp3 expression after naive T_{H} cells from IL-21 KO mice and their littermate control mice were activated with the indicated cytokines and neutralizing antibodies. Five days later, cells were assessed for Foxp3 expression by using intracellular staining. Numbers in quadrants are percentages.

FIG. 3C depicts mRNA expression of various genes as analyzed by real-time RT-PCR after naive T_{H} cells from IL-21 KO mice and their littermate control mice were activated with the indicated cytokines. The data shown are normalized to the expression of a reference gene, that encoding b-actin. Open columns, wild type; filled columns, IL-21 KO. The experiments were repeated three times with consistent results. WT, wild type.

FIG. 4A Il21^{+/+} (diamonds), Il21^{+/-} (squares) and Il21^{-/-} (triangles) mice were immunized with MOG peptide to induce EAE. Disease scores (means 6 s.e.m.) combining three independent experiments (wild type (WT), n=6 mice; Il21^{+/-}, n=17 mice; Il21^{-/-}, n=9 mice) are shown. P values were calculated with the Mann-Whitney U-test by comparing the disease score of WT and Il21^{-/-} mice and are indicated as followed: asterisk, P<0.05; two asterisks, P<0.005; three asterisks, P<0.001.

FIG. 4B depicts results when mononuclear cells isolated from spinal cords and brains (left) were stimulated for 5 h with TPA and ionomycin, and splenocytes (right) were re-stimulated with MOG peptide for 24 h, followed by intracellular staining of IL-17 and IFN-c and analysis in a CD4+ gate.

FIG. 5A shows results when OT-II T cells differentiated under indicated conditions were restimulated with anti-CD3 for 24 hours for cytokine measurement by ELISA.

FIG. 5B shows results when B6 and IL-6 KO mice (three each) were immunized with KLH in CFA. Seven days later, splenocytes were stimulated with various concentration of KLH and cytokines were measured by ELISA.

FIG. 5C shows results when naive T cells from wild-type (WT) and STAT3-deficient mice were activated as indicated. T cells were restimulated for 24 hours for IL-21 protein measurement. ELISA data are mean±s.d. for triplicates. The results shown are one representative of at least two independent experiments.

FIG. 5D shows results when naive T cells from wild-type (WT) and RORγ-deficient mice were activated as indicated. T cells were restimulated for 24 hours for IL-21 protein measurement. ELISA data are mean±s.d. for triplicates. The results shown are one representative of at least two independent experiments.

FIG. 6A shows results when naïve T cells from STAT3-deficient mice and their littermate controls were activated as in FIGS. 1D and 1E, respectively for 5 days. Following anti-CD3 restimulation, mRNA expression of indicated genes was analyzed by real-time RT-PCR. The RT-PCR data shown were normalized according to β-actin levels and expression in neutral conditions was set as 1. The results shown are one representative of at least two independent experiments.

FIG. 6B shows results when naïve T cells from RORγ-deficient mice and their littermate controls were activated as in FIGS. 1D and 1E, respectively for 5 days. Following anti-CD3 restimulation, mRNA expression of indicated genes was analyzed by real-time RT-PCR. The RT-PCR data shown were normalized according to β-actin levels and expression in neutral conditions was set as 1. The results shown are one representative of at least two independent experiments.

FIG. 7 illustrates the regulation of T_{H}17 differentiation by IL-21 in WT or IL-6 deficient T_{H} cells. Naïve T cells from B6 and IL-6 KO mice were activated as indicated in the presence of blocking anybodies to IL-4 and IFNγ. Five days later, T cells were assayed by intracellular staining.

FIG. 8A depicts enhanced Foxp3 expression in STAT3-deficient T_{H} cells following TH17 differentiation. Naïve T cells from STAT3 KO and their appropriate control mice were activated and analyzed as in FIGS. 2C and 2D, respectively. Numbers in dot plot quadrants represent the percentages. The data represent at least two independent experiments with consistent results.

FIG. 8B depicts enhanced Foxp3 expression in RORγ-deficient T_{H} cells following T_{H}17 differentiation. Naïve T cells from RORγ KO and their appropriate control mice were activated and analyzed as in FIGS. 2C and 2D, respectively. Numbers in dot plot quadrants represent the percentages. The data represent at least two independent experiments with consistent results.

FIG. 9 depicts normal development of T and B cells IL-21-deficient mice. FACS analysis of CD4+ and CD8+ T cells from thymus and CD4+ and CD8+ T cells and B220+ B cells from spleen of IL-21-deficient mice and littermate wild-type (WT) mice. Foxp3 expression was assessed in thymocytes by flow cytometry and analyzed in gated CD4 single positive cells. The expression of CD44 and CD62L was also analyzed on gated CD4+ T cells from spleen of WT and IL-21 deficient mice. Numbers in dot plot quadrants represent the percentages.

FIG. 10A illustrates that exogenous IL-21 restores T_{H}17 differentiation in IL-21-deficient T cells. Naïve T_{H} cells from IL-21 KO and their littermate control mice were activated with indicated cytokines and neutralizing antibodies. Five days later, cells were assessed for IFNγ and IL-17 production using intracellular staining. Numbers in quadrants represent the percentages. mRNA expression of various genes was analyzed by real-time RT-PCR.

FIG. 10B illustrates that exogenous IL-21 restores T_{H}17 differentiation in IL-21-deficient T cells. Naïve T_{H} cells from IL-21 KO and their littermate control mice were activated with indicated cytokines and neutralizing antibodies. Five days later, cells were assessed for IFNγ and Foxp3 expression using intracellular staining. Numbers in quadrants represent the percentages. mRNA expression of various genes was analyzed by real-time RT-PCR.

FIG. 11A illustrates that IL-21 deficiency impairs IL-17 expression in vivo. Lamina propria lymphocytes and spleen cells from IL-21 KO and their littermate control mice were analyzed for IL-17 expression in CD4+ and TCRγδ+ gates. The data represent at least two independent experiments with consistent results.

FIG. 11B illustrates that IL-21 deficiency impairs IL-17 expression in vivo. WT and IL-21 KO mice (three mice each) were immunized with MOG peptide in CFA. 7 days after immunizations, splenocytes were restimulated using PMA plus ionomycin or MOG peptide for intracellular staining or with MOG peptide for 4 days for ELISA analysis. Numbers in dot plot quadrants represent the percentages. For ELISA analysis, three mice were analyzed individually and the results shown are mean±s.d.

FIG. 12 depicts a revised scheme of T_{H} differentiation. T_{H} differentiation into effector lineages is regulated by innate cytokines (IL-12 and IL-27 for T_{H}1, IL-25 for T_{H}2, and IL-6 and TGFβ for T_{H}17 cells) and cytokines expressed by T cells (IFNγ for T_{H}1, IL-4 for T_{H}2 and IL-21 for T_{H}17 cells). Autocrine cytokines signal through selective STAT proteins to upregulate lineage-specific master regulators that result in terminal differentiation of T_{H} cells.

FIG. 13 depicts the results when CD4+ T cells from OT-II TcR transgenic mice were activated with Ova peptide and irradiated splenic APC in the presence of TGFβ, IL-6, IL-23, anti-IL-4, anti-IFNγ and in the presence or absence of anti mouse IL-21 antibodies. Five days later, cells were analyzed for IFNγ and IL-17 expression using intracellular staining.

FIG. 14 depicts the results when CD4+ T cells from OT-II TcR transgenic mice were activated with Ova peptide and irradiated splenic APC in the presence of TGFβ, IL-6, and IL-23, anti-IL-4, anti-IFNγ and in the presence or absence of recombinant mouse IL-21/Fc chimera. FACS-sorted naive T cells from B6 mice were activated with plate-bound anti-CD3, anti-CD28 and IL-2 in the presence of indicated above cytokines. Five days later, cells were analyzed for IFNγ and IL-17 expression using intracellular staining.

### DETAILED DESCRIPTION OF THE INVENTION

Methods of modulating T_{H}17 cell mediated immune response by inhibiting or inducing IL-21 signaling are provided. These methods also provide modulating the expression and/or activity of IL-21 to mediate T_{H}17 cell immune response including the differentiation of T_{H}17 cells.

Methods of treating an inflammatory disease or condition in a subject are further provided. These methods are useful for treating and/or preventing inflammatory diseases. The methods provided herein may also be used in diseases or conditions associated with inflammation. These methods of treatment include administering an agent to modulate IL-21 signaling, expression or activity in an amount effective to modulate the differentiation of T_{H}17 cells. Hence, a useful agent may modulate the production, growth or activity of the T_{H}17 cell. This agent may also modulate the expression of IL-17, IL17-F, or IL-22, and/or be used in combination with other agents. Further provided are methods of modulating the function and/or generation of T_{H}17 cells by use of an IL-21 antagonist and/or IL-21 agonist in an amount effective to inhibit or induce the differentiation of T_{H}17 cells and/or modulate the expression of IL-17, IL17-F, IL-22, and IL-21 by T_{H}17 cells.

Agents useful in connection with the methods described herein include generally small molecules, proteins, peptides, antibodies and antibody fragments, and small interfering RNAs or micro RNA. The agents designed to target IL-21 signaling, T_{H}17 cells, IL-21, IL-21 receptors, and CD+4 helper cells can be useful in modulating the expression and/or activity of IL-21 that mediates the T_{H}17 immune response.

IL-21 is an autocrine cytokine necessary in T_{H}17 differentiation. IL-21 is also cytokine that is expressed by T_{H}17 cells at both the mRNA and protein levels. Korn, T., et al., IL-21 Initiates an Alternative Pathway to Induce Proinflammatory T(H)17 Cells, 2007, Nature 448, 484-487; Zhou, L., et al., IL-6 Programs T(H)-17 Cell Differentiation by Promoting Sequential Engagement of the IL-21 and IL-23 Pathways, 2007, Nat Immunol 8, 967-974.

Methods of treating an inflammatory disease or condition by modulating T_{H}17 cell mediated immune response through inhibition or inducement of the activity of IL-21 include administering a therapeutically effective amount of IL-21 antagonist or agonist to an individual in need thereof are provided herein. By modulating the T_{H}17 cell mediated immune response, the expression of IL-17, IL-17F, IL-22, and/or IL-21 may also modulated.

CD4⁺ T helper (T_{H}) cells are regulators of adaptive immune responses, acting to coordinate the other cellular components of the immune system. After activation by antigen-presenting cells (APCs), antigen-specific T_{H} cells differentiate into effector cells that are specialized in terms of the cytokines that they secrete. T_{H}-cell differentiation from naïve CD4+ T-cells is mediated by lineage-specific transcription mechanisms. Effector T_{H} cells include the T_{H}17-, T_{H}1- and T_{H}2-cell lineages based on their cytokine expression profiles and immune regulatory function. Mosmann, T. R., et al., TH1 and TH2 Cells: Different Patterns of Lymphokine Secretion Lead to Different Functional Properties, 1989, Annu. Rev. Immunol. 7, 145-173. T_{H}-cell “lineages” are distinguishable based on the following criteria: first, that naive T_{H} cells differentiate independently into each subset in vitro and in vivo; and second, that each lineage has gene expression signatures that are distinct and heritable.

For example, T_{H}1 cells produce interferon-γ (IFNγ) and regulate cellular immunity. T_{H}2 cells produce interleukin-4 (IL-4), IL-5, and IL-13 and mediate humoral immunity and allergic responses. Studies have been conducted on the T_{H}1- and T_{H}2-cell subsets. Dong, C., et al., T_{H}1 and T_{H}2 Cells, 2001, Curr Opin Hematol 8, 47-51; Glimcher, L. H., et al., Lineage Commitment in the Immune System: The T Helper Lymphocyte Grows Up, 2000, Genes Dev. 14, 1693-1711.

Polarized T_{H}-cell differentiation into the T_{H}1- and T_{H}2-cell lineages was determined by the cytokine environment. T_{H}1 cells produce interferon-γ (IFNγ), and regulate antigen presentation and cellular immunity. Specifically, ligation of the T-cell receptor (TCR) and co-stimulatory receptors and interleukin-12 (IL-12) drives the process of T_{H}1-cell differentiation. IL-12 regulates T_{H}1-cell differentiation by activating the transcription factor signal transducer and activator of transcription 4 (STAT4). Trinchieri, G., et al., The IL-12 Gamily of Heterodimeric Cytokines: New Players in the Regulation of T Cell Responses, 2003, Immunity 19, 641-644. The transcription factors signal transducer and activator of transcription 4 (STAT4), T-bet, H2.0-like homeo box 1 (HLX1) and eomesodermin (EOMES) also have important regulatory functions in T_{H}1-cell differentiation. Szabo, S. J., et al., Molecular Mechanisms Regulating Th1 Immune Responses, 2003, Annu. Rev. Immunol, 21, 713-758. Signaling cascades induced by TCR crosslinking and IL-12 eventually lead to expression of the transcription factor T-bet, which is a master regulator of T_{H}1-cell differentiation because it potentiates the production of IFNγ and suppresses the expression of T_{H}2 cytokines.

On the other hand, T_{H}2 cells produce IL-4, IL-5 and IL-13, which are important regulators in humoral immunity and allergic responses. IL-4 is required for T_{H}2-cell differentiation. Glimcher, L. H., et al., Lineage Commitment in the Immune System: The T Helper Lymphocyte Grows Up, 2000, Genes Dev. 14, 1693-1711. The transcription factors STAT6, GATA-binding protein 3 (GATA3), nuclear factor of activated T cells, cytoplasmic 1 (NFATc1), MAF and JUNB regulate T_{H}2-cell differentiation and cytokine expression. IL-4 drives T_{H}2-cell differentiation through the action of STAT6, which upregulates expression of GATA-binding protein 3 (GATA3), a master regulator of T_{H}2-cell differentiation that is both necessary and sufficient for T_{H}2-cell development. Glimcher, L. H., et al., Lineage Commitment in the Immune System: The T Helper Lymphocyte Grows Up, 2000, Genes Dev. 14, 1693-1711; Zheng, W., et al., The Transcription Factor GATA-3 Is Necessary and Sufficient for Th2 Cytokine Gene Expression in CD4 T Cells, 1997, Cell 89, 587-596. In addition, MAF, which was identified as the first T_{H}2-cell-specific transcription factor that binds the Il4 proximal promoter, has an important role in IL-4 production once the T_{H}2-cell programme has been established. Ho, I. C., et al., The Proto-Oncogene C-MAF Is Responsible For Tissue-Specific Expression of Interleukin-4, 1996, Cell 85, 973-983.

Until recently T_{H} cells were thought to be a binary system, consisting of either T_{H}1 or T_{H}2 cells. As a result, T_{H}1- and T_{H}2-cell subsets have been used to explain certain T_{H}-cell function in immune responses and diseases. T_{H}2 cells have been found to be protective in organ-specific autoimmune diseases. On the other hand, T_{H}1 cells are pathogenic. Moreover, a deficiency in IFNγ or IL-12 does not abrogate, and can increase the onset and severity of disease as shown in mouse models of autoimmunity. Chu, C. O., et al., Failure to Suppress the Expansion of the Activated CD4 T Cell Population in Interferon γ-Deficient Mice Leads to Exacerbation of Experimental Autoimmune Encephalomyelitis, 2000, J. Exp. Med. 192, 123-128; Murphy, C. A., et al., Divergent Pro- and Anti-Inflammatory Roles for IL-23 and IL-12 in Joint Autoimmune Inflammation, 2003, J. Exp. Med. 198, 1951-1957.

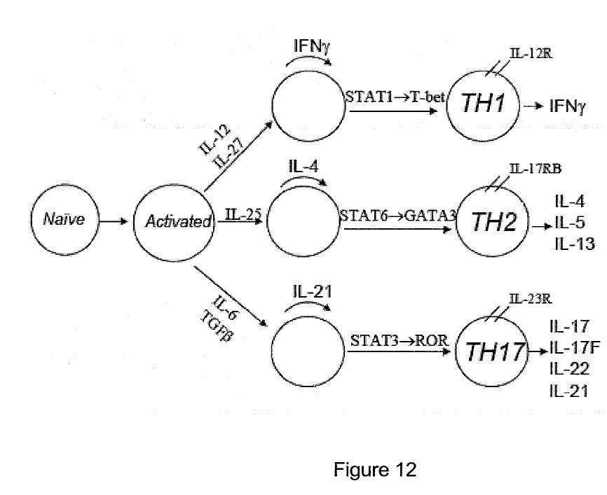

On the other hand, we have found that IL-21 regulates the differentiation of CD4⁺ T cells into T_{H}17 cells in an autocrine manner. Expression of IL-21 is induced in T cells by IL-6 via STAT3 and is necessary in the generation of T_{H}17 cells via STAT3-dependent upregulation of RORγ. IL-21 acts in an autocrine fashion in the differentiation of T_{H}17 cells as IFNγ does for T_{H}1 cells and IL-4 for T_{H}2 cells. FIG. 12 is a graphic depiction of T_{H} differentiation as now understood by the inventors.

According to this model shown in FIG. 12, IL-21, its signaling, expression and activity is now a target in treating a T_{H}17 cell mediated immune response. In addition, because IL-21 has regulatory functions in many immune responses, IL-17-expressing T cells (which produce large amounts of IL-21) are also a target for treating and preventing certain immune functions.

IL-21 belongs to the common gamma-chain family and regulates T, B, NK and dendritic cells. Leonard, W. J., et al., Interleukin-21: A Modulator of Lymphoid Proliferation, Apoptosis and Differentiation, 2005, Nature Rev. Immunol. 5, 688-698. IL-21 is mainly expressed by CD4+ T cells, and as previously shown, at higher levels by T_{H}2 than T_{H}1 cells. Wurster, A. L. et al., Interleukin 21 Is a T Helper (Th) Cell 2 Cytokine That Specifically Inhibits the Differentiation of Naïve Th Cells Into Interferon Gamma-Producing Th1 Cells, 2002, J Exp Med 196, 969-77. Addition of IL-21 during T_{H}1 differentiation can reduce IFNγ production through repressing Eomes expression.

Differentiation of T_{H}17 cells can be modulated via the IL-21 signaling pathway. Any interruption to the pathway upstream of STAT3 will prevent the activation of STAT3, the differentiation of T_{H}17 cells and ultimate expression of IL-21 and other cytokines expressed by the T_{H}17 cells. Examples of such modulators include STAT3 small molecule inhibitors and the like. Moreover, the IL-6-STAT3 pathway is important in inducing the expression of IL-21 and, therefore, can be modulated to inhibit or induce the production of IL-21. For example, IL-21 uses a JAK kinase to activate STAT3. STAT3 activates T_{H}17 cell differentiation. Therefore, IL-21 inhibitors can suppress specific cytokine signaling pathways and modulate the differentiation of TH17 cells.

Also, inhibition of IL-21 activity by reducing IL-21 binding to its receptor with a therapeutically effective amount of IL-21 antagonist such as an IL-21 blocking antibody can decrease the differentiation and/or affect the function of T_{H}17 cells. Functions of T_{H}17 cells which may be reduced include, for example and without limitation, expansion of T_{H}17 cells and production of IL-17, IL-17F, IL-22, and/or IL-21 by T_{H}17 cells. Inhibiting the expression of IL-21 serves to modulate the T_{H}17 cell mediated immune response.

IL-21 antagonists which are suitable agents to modulate T_{H}17 cell mediated immune response via inhibition of IL-21 activity include molecules that bind to IL-21 and/or the IL-21 receptor. Examples of IL-21 antagonists which may be suitable include soluble receptor antagonists and antibodies that prevent binding of IL-21 to the IL-21 receptor. IL-21 soluble receptors include, but are not limited to, recombinant chimeric proteins which bind to IL-21. IL-21 antagonists further include agents that bind to the IL-21 receptor to prevent any molecule from binding and/or activating the IL-21 receptor. Other types of suitable agents include siRNA and microRNA that down-regulates IL-21R expression or signaling, and inhibitors of IL-21 signaling components whose activity activates STAT3.

The IL-21 receptor consists of the IL-21R chain, and the common γ (γc)-chain, which is shared with the receptors for in humoral immunity and germinal-centre reactions, and provides the high expression of IL-21 by T_{FH} cells. Chtanova, T., et al., T Follicular Helper Cells Express a Distinctive Transcriptional Profile, Reflecting Their Role as Non-Th1/Th2 Effector Cells That Provide Help for B Cells, 2004, J Immunol 173, 68-78. In murine models, the anti mouse IL-21 antibody commercially available from R&D under catalog number AF594 have been shown to be effective in reducing T_{H}17 cell mediated immune response. In murine models, the recombinant mouse IL-21 R/Fc chimera commercially available from R&D under catalog number 596-MR has also been shown to be effective in reducing T_{H}17 cell mediated immune response.

Methods of modulating T_{H}17 cell mediated immune response through inhibition of IL-21 may be useful for the treatment of disorders or a wide variety of conditions where decreased T_{H}17 cell mediated immune response is useful. Disorders or conditions advantageously treated by these methods include inflammatory disease, including autoimmune diseases, which involve T_{H}17 cell mediated immune response. Examples of disorders or conditions which may be treated by decreasing T_{H}17 cell mediated immune response include, but are not limited to, inflammation, cancer, multiple sclerosis, arthritis, rheumatoid arthritis, asthma, systemic lupus erythematosus, allograft rejection, psoriasis, and inflammatory bowel disease. Further examples include ankylosing spondilitis, scleroderma, Type I diabetes, psoriatic arthritis, osteoarthritis, and atopic dermatitis.

Further disclosed herein are methods of treating infection by inducing T_{H}17 cell mediated immune response through induction of IL-21 activity and/or expression. These methods include administering a therapeutically effective amount of IL-21 or an IL-21 agonist to an individual in need thereof. Here, the IL-21 increases the generation, differentiation and function of T_{H}17 cells in the individual, thereby increasing T_{H}17-mediated response to infection or, in some cases, tumors. Examples of the types of infections or tumors that may benefit from increased T_{H}17 mediated immune response include immunity against excellular bacteria, fugus, viruses, and tumors including melanoma. IL-21 receptor agonist or similar receptor agonists including mimicry fragment, small molecule and molecules and proteins of similar function may be useful.

The phrase “therapeutically effective” is intended to qualify the amount of active ingredients used in the treatment of a disease or disorder. This amount will achieve the goal of reducing or eliminating the said disease or disorder.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

While it may be possible for the molecules which inhibit or induce IL-21 activity to be administered as the raw chemical, it is also possible to present them as a pharmaceutical formulation. Accordingly, the pharmaceutical formulation may include the molecule or a pharmaceutically acceptable salt, ester, prodrug or solvate thereof, where appropriate, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences.

The formulations of use molecules include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods may include the step of bringing into association the molecule or a pharmaceutically acceptable salt, ester, prodrug or solvate thereof (“active ingredient”) with the carrier which constitutes one or more accessory ingredients.

In certain instances, it may be appropriate to administer at least one molecule (or a pharmaceutically acceptable salt, ester, or prodrug thereof) in combination with another therapeutic agent. By way of example only, if one of the side effects experienced by a patient upon receiving one molecule is hypertension, then it may be appropriate to administer an anti-hypertensive agent in combination with the initial therapeutic agent. Or, by way of example only, the therapeutic effectiveness of one molecule may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one molecule as described herein with another therapeutic agent (which also includes a therapeutic regimen) that also has therapeutic benefit.

Multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Interleukin-17 (IL-17) Cytokines. IL-17 (also known as IL-17A) is the founding member of the IL-17 family of cytokines, which now has six members: IL-17A, IL-17B, IL-17C, IL-17D, IL-17E (also known as IL-25) and IL-17F. Aggarwal, S., et al., IL-17: Prototype Member of an Emerging Cytokine Family, 2002, J. Leukoc. Biol. 71, 1-8; Kolls, J. K., et al., Interleukin-17 Family Members and Inflammation, 2004, Immunity 21, 467-476; Park, H., et al., A Distinct Lineage of CD4 T Cells Regulates Tissue Inflammation by Producing Interleukin 17, 2005, Nat Immunol 6, 1133-1141; Moseley, T. A., et al., Interleukin-17 Family and IL-17 Receptors, 2003, Cytokine Growth Factor Rev 14, 155-174. The amino-acid sequence of IL-17 is most homologous to that of IL-17F and the genes encoding these two cytokines are located on the same chromosome in both the mouse and human. The genes encoding IL-17 and IL-17F are localized on human chromosome 6 and mouse chromosome 1.

IL-17 and IL-17F are expressed by CD4⁺ T cells and their expression is regulated by IL-23. Qian, Y., et al., The Adaptor Act1 Is Required for Interleukin 17-Dependent Signaling Associated With Autoimmune and Inflammatory Disease, 2007, Nat Immunol 8, 247-256. IL-17E is mainly expressed by T helper 2 (T_{H}2) cells and might have an important function in allergic responses. Shin, H. C. K., et al., Expression of IL-17 in Human Memory Cd45ro+T Lymphocytes and Its Regulation by Protein Kinase A Pathway, 1999, Cytokine 11, 257-266. IL-17, IL-17F and IL-22, which are produced by T_{H}17 cells, regulate the inflammatory responses of cells in non-lymphoid tissues, but do not regulate T-cell activation or differentiation. Harrington, L. E., et al., Interleukin 17-Producing CD4+ Effector T Cells Develop Via a Lineage Distinct From the T Helper Type 1 and 2 Lineages, 2005, Nat Immunol 6, 1123-1132; Chung, Y., et al., Expression and Regulation of IL-22 in the IL-17-Producing CD4+ T Lymphocytes, 2006, Cell Res 16, 902-7.

IL-17 Receptors. The receptor for IL-17 is IL-17R (also known as IL-17RA). Moseley, T. A., et al., Interleukin-17 Family and IL-17 Receptors, 2003, Cytokine Growth Factor Rev. 14, 155-174. Four other proteins share significant sequence homology with IL-17R, and are known as IL-17BR, IL-17RC, IL-17RD and IL-17RE. Moseley, T. A., et al., Interleukin-17 Family and IL-17 Receptors, 2003, Cytokine Growth Factor Rev. 14, 155-174. IL-17B and IL-17E both bind IL-17BR. The receptor for IL-17 is ubiquitously distributed in various tissues, and when engaged by its ligand(s) it induces activation of nuclear factor-κB (NF-κB) and JUN amino-terminal kinase (JNK) signalling pathways in a tumour-necrosis factor receptor (TNFR)-associated factor 6 (TRAF6)-dependent manner. Moseley, T. A., et al., Interleukin-17 Family and IL-17 Receptors, 2003, Cytokine Growth Factor Rev. 14, 155-174; Schwandner, R., et al., Requirement of Tumour Necrosis Factor Receptor-Associated Factor (TRAF)6 in Interleukin 17 Signal Transduction, 2000, J. Exp. Med. 191, 1233-1240.

T_{H}17 Cell Function. T_{H}17 cells are not only distinct in their gene expression and regulation, but also their biological function. T_{H}17 cells, particularly through the production of IL-17 and IL-17F, are pro-inflammatory. Dong, C., Diversification of T-Helper-Cell Lineages: Finding the Family Root of IL-17-Producing Cells, 2006, Nat Rev Immunol 6, 329-334; Kolls, J. K., et al., Interleukin-17 Family Members and Inflammation, 2004, Immunity 21, 467-476; Moseley, T. A., et al., Interleukin-17 Family and IL-17 Receptors, 2003, Cytokine Growth Factor Rev 14, 155-174.

On the other hand, T_{H}17 cells have an important role in host defense against infection, by recruiting neutrophils and macrophages to infected tissues. Human memory T_{H}17 cells react with antigens derived from Candida albicans, whereas memory T cells specific for Mycobacterium tuberculosis had T_{H}1-cell phenotype, suggesting that these two subsets have differential functions in immune responses to infectious pathogens. Acosta-Rodriguez, E. V., et al., Surface Phenotype and Antigenic Specificity of Human Interleukin 17-Producing T Helper Memory Cells, 2007, Nat Immunol 8, 639-646. IL-17 expressed by T_{H} cells has been associated with host defense against infectious agents and with autoimmune diseases. In host defense, IL-17 is important against bacterial infection in the mouse.

T_{H}17 cells are important in autoimmune and inflammatory diseases. Bettelli, E., et al., T(H)-17 Cells in the Circle of Immunity and Autoimmunity, 2007, Nat Immunol 8, 345-350; Steinman, L., A Brief History of TH17, The First Major Revision in the TH1/TH2Hypothesis of T Cell-Mediated Tissue Damage, 2007, Nat Med 13, 139-135. IL-21 is a potent regulator of CD8⁺ T-cell proliferation. Chtanova, T., et al., T Follicular Helper Cells Express a Distinctive Transcriptional Profile, Reflecting Their Role as Non-Th1/Th2 Effector Cells That Provide Help for B Cells, 2004, J Immunol 173, 68-78. Furthermore, T_{H}17-cell-derived IL-21 may be important for humoral immunity.

IL-17 and IL-17-producing cells regulate inflammatory responses. IL-17 expression is associated with several pro-inflammatory diseases in human, including rheumatoid arthritis, systemic lupus erythematosus, multiple sclerosis and asthma. Aggarwal, S., et al., IL-17: Prototype Member of an Emerging Cytokine Family, 2002, J. Leukoc. Biol. 71, 1-8; Kolls, J. K., et al., Interleukin-17 Family Members and Inflammation, 2004, Immunity 21, 467-476.

But IL-17 does not regulate T-cell function. Rather, IL-17 binds to other types of cells such as fibroblasts, epithelial cells and endothelial cells. IL-17 treatment of these cells induces the expression of pro-inflammatory cytokines such as IL-6 and granulocyte colony-stimulating factor (G-CSF), chemokines and matrix metalloproteinases. For example, IL-17 treatment of mouse fibroblasts leads to the activation of nuclear factor-κB (NF-κB) and mitogen-activated protein (MAP) kinases in a tumour-necrosis factor receptor (TNFR)-associated factor 6 (TRAF6)-dependent manner. Schwandner, R., et al., Requirement of Tumour Necrosis Factor Receptor-Associated Factor (TRAF)6 in Interleukin 17 Signal Transduction, 2000, J. Exp. Med. 191, 1233-1240.

Of the other IL-17-cytokine-family members, IL-17F has a similar function. Kolls, J. K., et al., Interleukin-17 Family Members and Inflammation, 2004, Immunity 21, 467-476. Therefore modulation of IL-21 activity to inhibit the generation and/or function of T_{H}17 cells, which produce IL-17 and IL-17F, can be used to treat or prevent these inflammatory immune responses.

Recently, the downstream targets of IL-17 have been also systematically analyzed by microarray analysis. Ruddy, M. J., et al., Functional Cooperation Between Interleukin-17 and Tumour Necrosis Factor-A Is Mediated by CCAAT/Enhancer-Binding Protein Family Members, 2004, J. Biol. Chem. 279, 2559-2567; Park, H., et al., A Distinct Lineage of CD4 T Cells Regulates Tissue Inflammation by Producing Interleukin 17, 2005, Nature Immunol. 6, 1133-1141. IL-17 and TNF function have been found synergistically to upregulate the expression of the gene encoding the pro-inflammatory cytokine IL-6 and the genes encoding the chemokines CXC-chemokine ligand 1 (CXCL1), CXCL2, CXCL5, CC-chemokine ligand 2 (CCL2) and CCL5 by the mouse osteoblastic cell line MC-3T3, as well as primary fibroblasts. Ruddy, M. J., et al., Functional Cooperation Between Interleukin-17 and Tumour Necrosis Factor-A Is Mediated by CCAAT/Enhancer-Binding Protein Family Members, 2004, J. Biol. Chem. 279, 2559-2567; Shen, F., et al., Cytokines Link Osteoblasts and Inflammation: Microarray Analysis of Interleukin-17- and TNF-A-Induced Genes in Bone Cells, 2005, J. Leukoc. Biol. 77, 388-399. In another study, Park et al. found that the genes encoding several chemokines (CCL2, CCL7, CXCL1 and CCL20), as well as the genes encoding matrix metalloproteinase 3 (MMP3) and MMP13 were substantially upregulated in mouse embryonic fibroblasts following treatment with IL-17. Park, H., et al., A Distinct Lineage of CD4 T Cells Regulates Tissue Inflammation by Producing Interleukin 17, 2005, Nature Immunol 6, 1133-1141.

Similarly, in in vivo studies, IL-17 has been shown to regulate inflammatory responses. IL-17-receptor-deficient mice showed impaired host defense against infection with Klebsiella pneumoniae owing to a substantial reduction in the amount of G-CSF and CXCL2 in the lung, and therefore a marked decrease in neutrophil recruitment. Ye, P., et al., Requirement of Interleukin 17 Receptor Signaling for Lung CXC Chemokine and Granulocyte Colony-Stimulating Factor Expression, Neutrophil Recruitment, and Host Defense, 2001, J. Exp. Med. 194, 519-527. Conversely, overexpressing IL-17 in the lungs resulted in chemokine upregulation and tissue infiltration by leukocytes. Park, H., et al., A Distinct Lineage of CD4 T Cells Regulates Tissue Inflammation by Producing Interleukin 17, 2005, Nature Immunol 6, 1133-1141. Recently, overproduction of IL-17 was also found to be functionally associated with hepatic granulomatous inflammation. Rutitzky, L. I., et al., Severe CD4 T Cell-Mediated Immunopathology in Murine Schistosomiasis Is Dependent on IL-12p40 and Correlates With High Levels of IL-17, 2005, J. Immunol. 175, 3920-3926.

Moreover, in adjuvant-induced models of rheumatoid arthritis, IL-17-deficient mice, as well as mice administered an IL-17-receptor antagonist, were found to be resistant to disease onset. Nakae, S., et al., Suppression of Immune Induction of Collagen-Induced Arthritis in IL-17 Deficient Mice, 2003, J. Immunol. 171, 6173-6177; Bush, K. A., et al., Reduction of Joint Inflammation and Bone Erosion in Rat Adjuvant Arthritis by Treatment With Interleukin-17 Receptor Igg1 Fc Fusion Protein, 2002, Arthritis Rheum. 46, 802-805.

Further, evidence of a role for IL-17 in the pathogenesis of autoimmune disease was provided by studies with animals deficient for inducible T-cell co-stimulator (ICOS)—ICOS-deficient mice were found to show resistance to collagen-induced arthritis (CIA) and this was associated with a selective deficiency in IL-17 expression. Dong, C., et al., Regulation of Immune and Autoimmune Responses by ICOS, 2003, J. Autoimmun. 21, 255-260. Recently, mice treated with a neutralizing IL-17-specific antibody were also found to be resistant to the induction of EAE. Park, H., et al., A Distinct Lineage of CD4 T Cells Regulates Tissue Inflammation by Producing Interleukin 17, 2005, Nature Immunol 6, 1133-1141; Langrish, C. L., et al., IL-23 Drives a Pathogenic T Cell Population That Induces Autoimmune Inflammation, 2005, J. Exp. Med. 201, 233-240.

In one of these studies, Park et al. found that the action of the IL-17-specific antibody did not cause defective autoreactive T-cell activation or cytokine expression but was instead associated with inhibition of chemokine expression in the brain. Park, H., et al., A Distinct Lineage of CD4 T Cells Regulates Tissue Inflammation by Producing Interleukin 17, 2005, Nature Immunol 6, 1133-1141. Hence, IL-17 exerted its pro-inflammatory function through regulation of chemokine expression, and is different than IFNγ and IL-4 in regulating cellular and humoral immunity.

Cellular and Molecular Expression of IL-17 by T_{H}17 cells. IL-17 has been shown to be important in several animal models of autoimmunity and its expression is often associated with human inflammatory diseases. IL-17 has been found to be expressed by CD8⁺ T cells, γδ T cells, and neutrophils. Shin, H. C. K., et al., Regulation of IL-17, IFN-γ and IL-10 in Human CD8 T Cells by Cyclic AMP-Dependent Signal Transduction Pathway, 1998, Cytokine 10, 841-850; Stark, M. A., et al., Phagocytosis of Apoptotic Neutrophils Regulates Granulopoiesis Via IL-23 and IL-17, 2005, Immunity 22, 285-294; Ferretti, S., et al., IL-17, Produced by Lymphocytes and Neutrophils, Is Necessary for Lipopolysaccharide-Induced Airway Neutrophilia: IL-15 as a Possible Trigger, 2003, J. Immunol. 170, 2106-2112. Neutrophils and γδ T cells can produce IL-17 rapidly in an early phase of immune responses against infection and thereby regulate innate inflammatory responses, such as neutrophil or macrophage infiltration into infected tissues.

IL-17-producing cells are a subset of CD4⁺ T cells that produce IL-17. Initially, IL-17 was found to be expressed by activated human CD4⁺ T cells and by T_{H}1- and T_{H}0-cells but not by T_{H}2 cells. Yao, Z., et al., IL-17: A Novel Lcytokinen Derifed From T Cells, 1995, J. Immunol. 155, 5483-5486. Subsequently, Infante-Duarte et al. found that IL-17 expression by mouse CD4⁺ T cells was induced in the presence of microbial lipopeptide. Infante-Duarte, C., et al., Microbial Lipopeptides Induce the Production of IL-17 in Th Cells, 2000, J. Immunol. 165, 6107-6115. CD4⁺ T cells that produce IL-17 also produce TNF but not IFNγ, indicating that IL-17-producing cells might represent a unique T_{H}-cell subset or activation state. Infante-Duarte, C., et al., Microbial Lipopeptides Induce the Production of IL-17 in Th Cells, 2000, J. Immunol. 165, 6107-6115.

As noted above, IL-17-producing T_{H} cells are distinct from traditional T_{H}1 or T_{H}2 cells in their cytokine expression profile and immune function. Two studies that analyzed the molecular pathways that direct IL-17 expression by CD4⁺ T cells clearly indicate that IL-17-producing CD4⁺ T cells represent a distinct lineage of T_{H} cells that develop independently of T_{H}1 and T_{H}2 cytokines and the transcription factors that regulate the differentiation and maintenance of the T_{H}1 and T_{H}2-cell subsets. Park, H., et al., A Distinct Lineage of CD4 T Cells Regulates Tissue Inflammation by Producing Interleukin 17, 2005, Nature Immunol 6, 1133-1141; Harrington, L. E. et al., Interleukin 17-Producing CD4⁺ Effector T Cells Develop Via a Lineage Distinct From the T Helper Type 1 and 2 Lineages, 2005, Nature Immunol. 6, 1123-1132.

Not only do IL-17-producing CD4⁺ T cells express distinct cytokines from those produced by T_{H}1 cells and T_{H}2 cells, but naïve CD4⁺ T cells develop into this lineage in vitro and in vivo through a pathway that is independent of the programs governing T_{H}1- and T_{H}2-cell differentiation. The antigen-specific naive CD4⁺ T cells differentiate poorly in vitro into IL-17-expressing cells, even in the presence of IL-23. Park, H. et al., A Distinct Lineage of CD4 T Cells Regulates Tissue Inflammation by Producing Interleukin 17. 2005, Nature Immunol. 6, 1133-1141; Harrington, L. E. et al., Interleukin 17-Producing CD4+ Effector T Cells Develop Via a Lineage Distinct From the T Helper Type 1 and 2 Lineages, 2005, Nature Immunol. 6, 1123-1132. However, neutralizing IFNγ using an IFNγ-specific antibody greatly potentiated the differentiation of naive CD4⁺ T cells into IL-17-producing cells, showing IFNγ as a negative regulator of this process. Park, H., et al., A Distinct Lineage of CD4 T Cells Regulates Tissue Inflammation by Producing Interleukin 17, 2005, Nature Immunol 6, 1133-1141; Harrington, L. E. et al., Interleukin 17-Producing CD4⁺ Effector T Cells Develop Via a Lineage Distinct From the T Helper Type 1 and 2 Lineages, 2005, Nature Immunol. 6, 1123-1132.

Type 1 IFNs also have been shown to inhibit the generation of IL-17-producing cells. Harrington, L. E. et al., Interleukin 17-Producing CD4⁺ Effector T Cells Develop Via a Lineage Distinct From the T Helper Type 1 and 2 Lineages, 2005, Nature Immunol. 6, 1123-1132. In the presence of neutralizing IFNγ-specific antibody, T_{H}2-cell differentiation was also increased, but treatment with neutralizing IL-4-specific antibody and IFNγ-specific antibody synergistically increased the number of IL-17-producing cells. Park, H., et al., A Distinct Lineage of CD4 T Cells Regulates Tissue Inflammation by Producing Interleukin 17, 2005, Nature Immunol 6, 1133-1141; Harrington, L. E. et al., Interleukin 17-Producing CD4⁺ Effector T Cells Develop Via a Lineage Distinct From the T Helper Type 1 and 2 Lineages, 2005, Nature Immunol. 6, 1123-1132.

As noted above, IL-17-producing T cells are distinct from T_{H}1 and T_{H}2 cells. The regulation of cytokine expression by these cells is different than T_{H}17 cells. For example, IL-23 and IL-27 are two members of the IL-12 family. IL-12, a heterodimeric cytokine consisting of p35 and p40 subunits, is an important regulator of T_{H}1-cell differentiation. Trinchieri, G., et al., The IL-12 Family of Heterodimeric Cytokines: New Players in the Regulation of T Cell Responses, 2003, Immunity 19, 641-644; Hunter, C. A., New IL-12 Family Members: IL-23 and IL-27, Cytokines With Divergent Functions, 2005, Nature Rev. Immunol. 5, 521-531. IL-23 is a heterodimer that contains the same p40 subunit as IL-12 combined with a unique p19 subunit. Similar subunit sharing occurs for the receptors for IL-12 and IL-23. Similarly, the IL-12 receptor is a heterodimer composed of IL-12Rβ1 and IL-12Rβ2. The IL-23 receptor also contains IL-12Rβ1, but in combination with a specific receptor known as IL-23R. Trinchieri, G., et al., The IL-12 Family of Heterodimeric Cytokines: New Players in the Regulation of T Cell Responses, 2003, Immunity 19, 641-644.

IL-17 expression by mouse memory CD4⁺ T cells has been shown to be strongly induced by IL-23. Aggarwal, S., et al., Interleukin-23 Promotes a Distinct CD4 T Cell Activation State Characterized by the Production of Interleukin-17, 2003, J. Biol. Chem. 278, 1910-1914. Moreover, mice deficient in IL-23 but not IL-12 show resistance to EAE and CIA. Cua, D. J., et al., Interleukin-23 Rather Than Interleukin-12 Is the Critical Cytokine for Autoimmune Inflammation of the Brain, 2003, Nature 421, 744-748; Murphy, C. A., et al., Divergent Pro- and Antiinflammatory Roles for IL-23 and IL-12 in Joint Autoimmune Inflammation, 2003, J. Exp. Med. 198, 1951-1957. Resistance to disease onset correlated with a defect in IL-17 expression has also been shown. Murphy, C. A., et al., Divergent Pro-and Antiinflammatory Roles for IL-23 and IL-12 in Joint Autoimmune Inflammation, 2003, J. Exp. Med. 198, 1951-1957; Langrish, C. L., et al., IL-23 Drives a Pathogenic T Cell Population That Induces Autoimmune Inflammation, 2005, J. Exp. Med. 201, 233-240. Hence, it is IL-23 that regulates IL-17 production by memory CD4⁺ T cells in mouse models of autoimmunity and not IL-12.

IL-23 has been found to be important in regulating IL-17 expression in anti-bacterial immunity. Happel, K. I., et al., Divergent Roles of IL-23 and IL-12 in Host Defense Against Klebsiella Pneumoniae, 2005, J. Exp. Med. 202, 761-769; Khader, S. A., et al., IL-23 Compensates for the Absence of IL-12p70 and Is Essential for the IL-17 Response During Tuberculosis But Is Dispensable for Protection and Antigen-Specific IFN-γ Responses if IL-12p70 Is Available, 2005, J. Immunol. 175, 788-795.

In addition, IL-23 has been reported to selectively induce the clonal expansion of antigen-primed IL-17-producing CD4⁺ T cells. Murphy, C. A., et al., Divergent Pro- and Antiinflammatory Roles for IL-23 and IL-12 in Joint Autoimmune Inflammation, 2003, J. Exp. Med. 198, 1951-1957; Langrish, C. L., et al., IL-23 Drives a Pathogenic T Cell Population That Induces Autoimmune Inflammation, 2005, J. Exp. Med. 201, 233-240. After immunization of mice with protein antigen, antigen-specific IL-17-producing CD4⁺ T cells could be expanded by restimulation of spleen and lymph node cells in vitro with the same antigen in the presence of IL-23.

By contrast, IL-12 potently increases the number of IFNγ-producing cells. IL-23 and IL-12 could increase the number of IL-17- and IFNγ-producing cells, respectively, from immunized p40-deficient animals indicating that these cytokines might not be absolutely required for CD4⁺ T cells to commit to making a particular cytokine. Murphy, C. A., et al., Divergent Pro- and Antiinflammatory Roles for IL-23 and IL-12 in Joint Autoimmune Inflammation, 2003, J. Exp. Med. 198, 1951-1957. When transferred to EAE-susceptible mice, CD4⁺ T cells cultured in vitro with antigen in the presence of IL-23 but not by IL-12 were pathogenic further supporting a role for IL-17-producing cells in causing autoimmune disease. Langrish, C. L., et al., IL-23 Drives a Pathogenic T Cell Population That Induces Autoimmune Inflammation, 2005, J. Exp. Med. 201, 233-240.

As a result of in vitro culture in the presence of neutralizing IFNγ-specific antibody and IL-23 the differentiated cells greatly upregulated expression of Il17 and Il17f mRNA, and downregulated expression of the genes encoding T-bet, eomesodermin (EOMES), and H2.0-like homeo box 1 (HLX1), indicating that CD4⁺ T-cell differentiation into IL-17-producing cells might not be mediated by the T_{H}1-cell transcriptional machinery. Harrington, L. E., et al., Interleukin 17-Producing CD4⁺ Effector T Cells Develop Via a Lineage Distinct From the T Helper Type 1 and 2 Lineages, 2005, Nature Immunol. 6, 1123-1132; C.D. unpublished data.

CD4⁺ T-cell differentiation into IL-17-producing cells was increased in the absence of STAT1, STAT6 and T-bet. Harrington, L. E., et al., Interleukin 17-Producing CD4⁺ Effector T Cells Develop Via a Lineage Distinct From the T Helper Type 1 and 2 Lineages, 2005, Nature Immunol. 6, 1123-1132. T_{H}2-cell transcription factor MAF has been shown to have a role in negative regulation of IL-17 expression. Park, H., et al., A Distinct Lineage of CD4 T Cells Regulates Tissue Inflammation by Producing Interleukin 17, 2005, Nature Immunol. 6, 1133-1141.

In vivo generation (differentiation) of IL-17-producing cells was produced following immunization with a peptide derived from myelin oligodendrocyte glycoprotein (MOG). Here, an IL-17-producing MOG-specific CD4⁺ T-cell population was induced. Park, H., et al., A Distinct Lineage of CD4 T Cells Regulates Tissue Inflammation by Producing Interleukin 17, 2005, Nature Immunol. 6, 1133-1141. Expression of IL-17 by MOG-specific CD4⁺ T cells occurred in mice deficient in IFNγ and in mice lacking the transcription factors STAT4, STAT6 or T-bet. Park, H., et al., A Distinct Lineage of CD4 T Cells Regulates Tissue Inflammation by Producing Interleukin 17, 2005, Nature Immunol. 6, 1133-1141. Therefore, the data shows that in vitro and in vivo, naive CD4⁺ T cells can directly differentiate into IL-17-producing cells, a process that is augmented by IL-23, but that is independent of T_{H}1 and T_{H}2 cytokines and the transcription factors mediating T_{H}1- and T_{H}2-cell differentiation.

### EXAMPLES

### Example 1

### General Methods

The examples disclosed herein generally utilized the following materials and utilized the following methods, as appropriate to the particular example:

Mice. IL-6 deficient mice were purchased from Jackson Laboratories, and IL-21 knockout mice came from NIH Mutant Mouse Regional Resource Centers (MMRRC). RORγ KO mice were backcrossed six generations onto C57BL/6. Stat3 fl and Tie2-Cre mice were bred to yield fl/Δ Cre+ and Cre−littermates as described by Yang, X. O., et al., STAT3 Regulates Cytokine-Mediated Generation of Inflammatory Helper T Cells, 2007, J. Biol. Chem. 282, 9358-9363. The animal experiments were performed using protocols approved by Institutional Animal Care and Use Committee.

IL-6 deficient mice on C57BL/6 background were purchased from Jackson Laboratories and C57BL/6 mice were used as controls. RORγ KO mice as previously reported by Kurebayashi et al. were backcrossed 6-7 generations onto C57BL/6 background and splenocytes from these and their littermate control mice were used for in vitro differentiation. Kurebayashi, S., et al., Retinoid-Related Orphan Receptor Gamma (RORgamma) Is Essential for Lymphoid Organogenesis and Controls Apoptosis During Thymopoiesis, 2000, Proc Natl Acad Sci USA97, 10132-7. STAT3 fl and Tie2-Cre mice were bred on 129×C57BL/6 mixed background to yield fl/Δ Cre+ and Cre−littermates as described by Yang, X. O. et al, and their spleen and lymph node cells were used for in vitro differentiation. IL-21 knockout mice on 129×C57BL/6 F1 mixed background were obtained from NIH Mutant Mouse Regional Resource Centers (MMRRC). Yang, X. O., et al., STAT3 Regulates Cytokine-Mediated Generation of Inflammatory Helper T Cells, 2007, J. Biol. Chem. 282, 9358-9363. IL-21+/− mice were intercrossed to generate Il21^{+/+}, Il21^{30 /-} and Il21^{-/-} mice for the experiments including EAE. Mice (except the ROR-γ KO mice) were housed in the SPF animal facility at M. D. Anderson Cancer Center and the animal experiments were performed at the age of 6-10 weeks using protocols approved by Institutional Animal Care and Use Committee.

T cell isolation and differentiation. Naïve CD4+CD25−CD62LhiCD44lo T cells were activated with plate-bound 2 μg/ml anti-CD3 and 2 μg/ml anti-CD28 and 50 units/ml IL-2 in the presence of 30 ng/ml IL-6 (Peprotech), 80 ng/ml IL-21, 50 ng/ml IL-23 (R&D system), 2.5 ng/ml TGFβ (Peprotech), 10 μg/ml anti-IL-4 (11B11), 10 μg/ml anti-IFNγ (XMG 1.2), 10 ng/ml TNFα, 10 ng/ml IL-1β or combination of these stimuli.

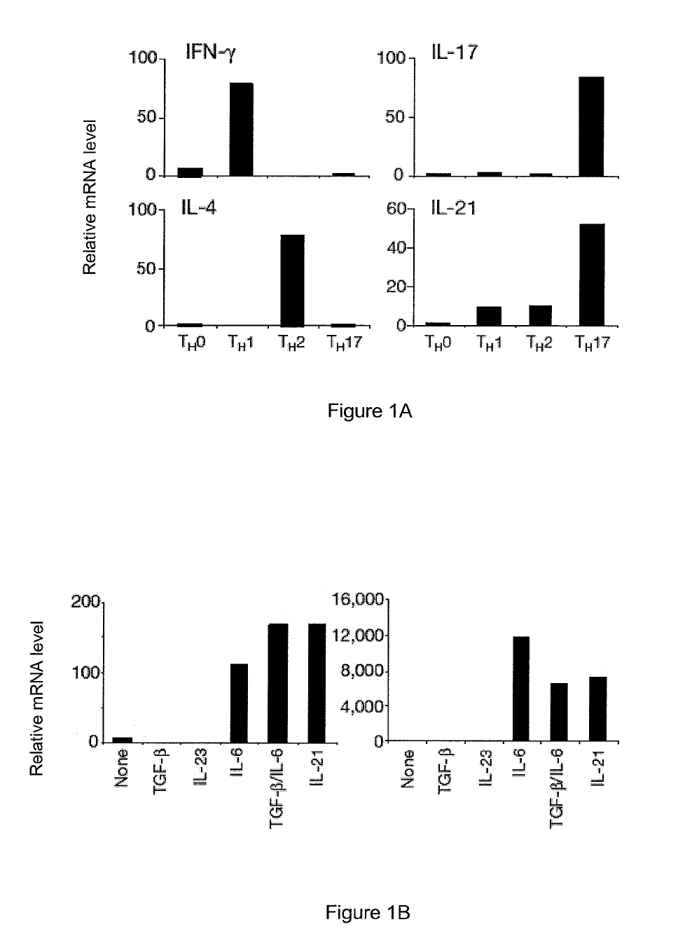

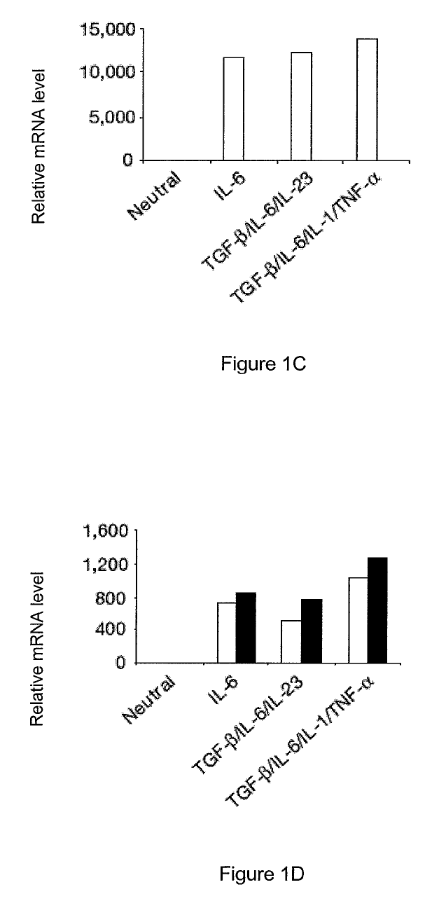

Differentiation of OT-II cells in FIG. 1A was performed as previously described. Chung, Y., et al., Expression and Regulation of IL-22 in the IL-17 Producing CD4⁺ T Lymphocytes, 2006, Cell Res. 16, 902-907. For naïve T cell differentiation in other experiments, CD4+CD25−CD62LhiCD44lo cells were FACS-sorted as described by Yang, X. O., et al., STAT3 Regulates Cytokine-Mediated Generation of Inflammatory Helper T Cells, 2007, J. Biol. Chem. 282, 9358-9363. Naïve CD4+ T cells were activated with plate-bound 2 μg/ml anti-CD3 and 2 μg/ml anti-CD28 and 50 units/ml IL-2 in the presence of 30 ng/ml IL-6 (Peprotech), 80 ng/ml IL-21, 50 ng/ml IL-23 (R&D system), 2.5 ng/ml TGFβ (Peprotech), 10 μg/ml anti-IL-4 (11B11), 10 μg/ml anti-IFNγ (XMG 1.2), 10 ng/ml TNFα, 10 ng/ml IL-1β or combination of these stimuli. Four to five days after activation, cells were washed and restimulated with PMA and ionomycin in the presence of Golgi-stop for 5 hours, after which IL-17- and IFNγ-producing cells were analyzed using intracellular staining. Intracellular staining for Foxp3 was performed by using a Foxp3 staining kit (eBioscience). Lymphocytes were isolated from small intestine lamina propria as previously described by Laky et al. and restimulated with PMA and ionomycin in the presence of Golgi-stop for 5 hours, after which IL-17-producing cells were analyzed using intracellular staining. Laky, K., et al., Age-Dependent Intestinal Lymphoproliferative Disorder Due to Stem Cell Factor Receptor Deficiency: Parameters in Small and Large Intestine, 1997, J. Immunol. 158, 1417-1427.

EAE induction. For the induction of EAE, mice were immunized with the MOG peptide emulsified in CFA. Mice were immunized subcutaneously at the dorsal flanks with 150 μg of MOG peptide in CFA at day 0 and day 7. Pertussis toxin was given intraperitoneally at day 1 and day 8 with the dosage of 500 ng per mouse. Signs of EAE were assigned scores on a scale of 1-5 as follows: 0, none; 1, limp tail or waddling gait with tail tonicity; 2, wobbly gait; 3, hind limb paralysis; 4, hind limb and forelimb paralysis; 5, death.

For the induction of EAE, female mice were immunized with the MOG peptide emulsified in CFA. Mice were immunized subcutaneously at the dorsal flanks with 150 μg of MOG peptide in CFA at day 0 and day 7. Pertussis toxin was given intraperitoneally at day 1 and day 8 with the dosage of 500 ng per mouse. Signs of EAE were assigned scores on a scale of 1-5 as follows: 0, none; 1, limp tail or waddling gait with tail tonicity; 2, wobbly gait; 3, hind limb paralysis; 4, hind limb and forelimb paralysis; 5, death. Disease scores from three independent experiments (WT, n=6 mice; Il21^{+/}−, n=17 mice; Il21^{-/-}, n=9 mice) were combined and p values calculated using the Mann-Whitney U test by comparing the disease scores of WT and Il21^{-/-} mice and Il21^{+/-} and Il21^{-/-} mice were found to be significant (<0.05). To analyze central nervous system infiltrates, both brain and spinal cord were collected from perfused mice and mononuclear cells were prepared by percoll gradient.

Immunizations. IL-6 knockout and C57BL/6 mice (6-8 wk old; three per group) were immunized with KLH (0.5 mg/ml) emulsified in CFA (0.5 mg/ml) at the base of the tail (100 μl each mouse). 7 days later, spleen cells from KLH-immunized mice were stimulated with or without KLH for 3 days, and effector cytokines (IFN-γ, IL-4, IL-17 and IL-21) were measured by ELISA (Pharmingen). IL-21 knockout mice and their littermate controls (three per group) were immunized with MOG peptide (amino acids 35-55; MEVGWYRSPFS ROVHLYRNGK) in CFA. Seven days later, spleen cells from immunized mice were restimulated with 50 ng/ml PMA and 500 ng/ml ionomycin (Sigma-Aldrich) for 5 h, or with 25 μg MOG peptide for 24 h. In the final 5 h, Golgi-stop (BD Bioscience) was added and IL-17- and IFNγ-producing cells were analyzed using a BD CytoFix/CytoPerm intracellular staining kit (BD Bioscience). In the same experiment, spleen cells were restimulated with or without MOG peptide for 3 days, and cytokine production was measured by ELISA.

Quantitative real-time PCR. Total RNA was prepared from T cells using TriZol regent (Invitrogen). cDNA were synthesized using Superscript reverse transcriptase and oligo(dT) primers (Invitrogen) and gene expression was examined with a Bio-Rad iCycler Optical System using the iQ™ SYBR green real-time PCR kit available from Bio-Rad Laboratories, Inc. The data were normalized to β-actin reference. The following primer pair for IL-21 was used: forward, TCATCATTGACCTCGTGGCCC, reverse, ATCGTACTTCTCCACTTGCAATCCC. The primers for IL-17, IL-17F, IL-23R, RORγ, IL-22, T-bet, Foxp3, and β-actin were previously described in Yang, X. O., et al., STAT3 Regulates Cytokine-Mediated Generation of Inflammatory Helper T Cells, 2007, J. Biol. Chem. 282, 9358-9363.

### Example 2

### Expression of IL-21 by T_{H}17 Cells

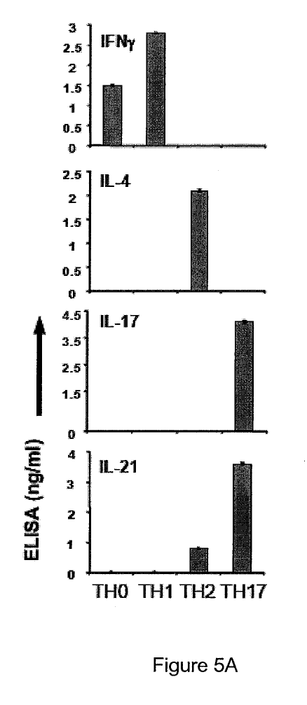

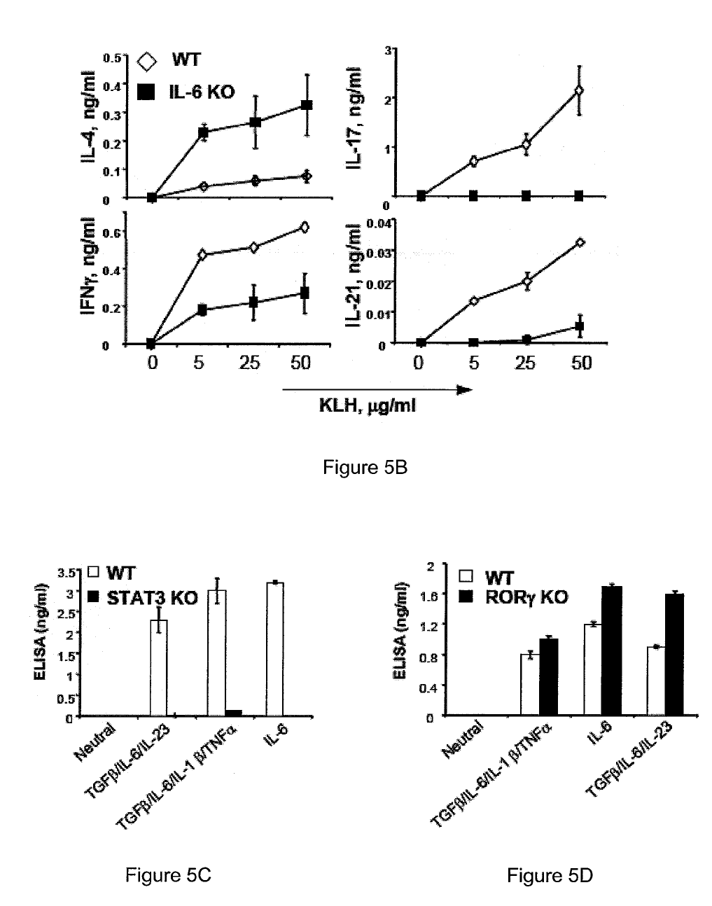

In a gene expression analysis of in-vitro differentiated T_{H}1, T_{H}2 and T_{H}17 cells, IL-21 expression was found to be increased in T_{H}17 cells compared to T_{H}1 or T_{H}2 cells (data not shown). However, IL-21 expression by T_{H}17 cells has not been reported. In OT-II cells activated under neutral (T_{H}0), T_{H}1 (IL-12+anti-IL-4), T_{H}2 (IL-4+anti-IFNγ) and T_{H}17 (IL-6, TGFβ, IL-23, anti-IFNγ and anti-IL-4) conditions, IL-17 and IL-21 mRNA expression were found to be significantly higher in T_{H}17 cells than other effector cells, as apparent in FIG. 1A. Consistently in the supernatants of T_{H}0, T_{H}1, T_{H}2 and T_{H}17 cells following anti-CD3 restimulation, although also produced by T_{H}2 cells, IL-21 secretion was significantly increased in T_{H}17 cells (FIG. 5A). These data indicate IL-21 as another cytokine highly produced by T_{H}17 cells.

### Example 3

### Regulation of IL-21 Expression During T_{H}17 Differentiation

To assess the regulation of IL-21 expression during T_{H}17 differentiation, naïve CD4+CD25−CD62LhiCD44lo T_{H} cells from C57BL/6 (B6) mice were FACS-sorted and activated with plate-bound anti-CD3 and anti-CD28 in the presence of various cytokine stimuli. Analyzed on day 2 and 5 post activation, IL-21 mRNA was upregulated by IL-6, but not TGFβ or IL-23, as apparent in FIG. 1B. No synergistic effect of TGFβ and IL-6 was observed, distinct from the regulation of IL-17 and IL-17F, where TGFβ and IL-6 exhibited a remarkable synergy, possibly via chromatin remodeling at the chromosomal locus containing these two highly homologous genes. Veldhoen, M., et al., TGFβ in the Context of an Inflammatory Cytokine Millieu Supports de novo Differentiation of IL-17 Producing T Cells, 2006, Immunity 24, 179-189; Akimzhanov, A. M., et al., Chromatin Remodeling of Interleukin-17 (IL-17)-IL-17F Cytokine Gene Locus During Inflammatory Helper T Cell Differentiation, 2007, J. Biol. Chem. 282, 5969-5972. FIG. 1B illustrates that IL-21 also increased its own mRNA expression, indicating an autocrine regulation.

### Example 4

### The Role of IL-6 in IL-21 Expression In Vivo

To examine the requirement of IL-6 in IL-21 expression in vivo, B6 and IL-6 knockout mice were immunized with keyhole limpet hemocyanin (KLH) in complete Freund's adjuvant (CFA). One week later, spleen cells from immunized mice were restimulated with KLH ex vivo. Enhanced IL-4 and reduced IFNγ production was observed in the supernatants of IL-6-deficient cells when compared to wild-type cells (FIG. 5B), consistent with previous literature. Okuda, Y., et al., IL-6 Plays a Crucial Role in the Induction Phase of Myelin Oligodendrocyte Glucoprotein 35-55 Induced Experimental Autoimmune Encephalomyelitis, 1999, J. Neuroimmunol, 101, 188-196; Okuda, Y., et al., Enhancement of Th2 Response in IL-6-Deficient Mice Immunized With Myelin Oligodendrocyte Glycoprotein, 2000, J. Neuroimmunol. 105, 120-123. IL-6-deficient cells did not produce IL-17 or IL-21. These data indicate that IL-6 is sufficient and necessary in inducing IL-21 production by T_{H} cells.

### Example 5

### The Role of STAT3 and RORγ in Expression of IL-21

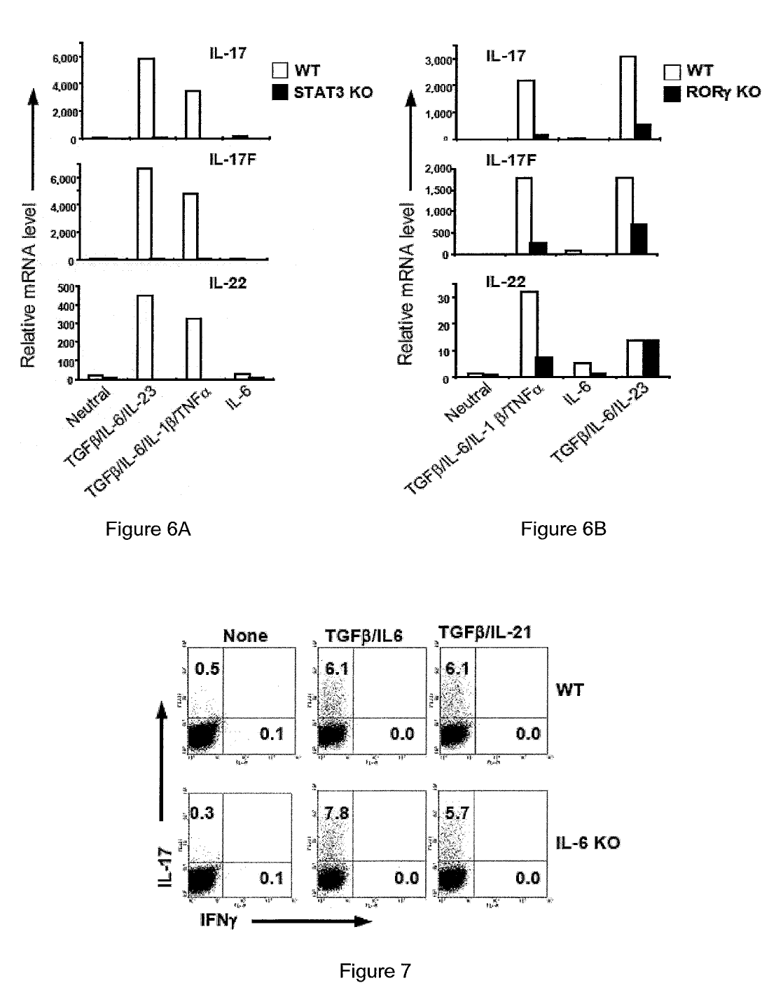

STAT3 and the RORγt isoform of RORγ are key transcription factors in T_{H}17 differentiation. Dong, C., Diversification of T-Helper-Cell Lineages: Finding the Family Root of IL-17-Producing Cells, 2006, Nature Rev. Immunol. 6, 329-334. To determine their regulation in IL-21 expression, naive T_{H} cells from STAT3-, or RORγ-deficient mice and their controls were activated as above. Dong, C., Diversification of T-Helper-Cell Lineages: Finding the Family Root of IL-17-Producing Cells, 2006, Nature Rev. Immunol. 6, 329-334. STAT3-deficient TH cells activated under all conditions failed to produce IL-21 mRNA or protein (FIGS. 1C and 5C), as well as IL-17, IL-17F and IL-22 (FIG. 6A). On the other hand, although Rorc^{-/-} T_{H} cells exhibited a severe deficiency in IL-17, IL-17F and IL-22 expression (FIG. 6B), IL-21 mRNA and protein expression were normal in these cells compared to those from wild-type mice (FIGS. 1D and 5D). All these data indicate that whereas STAT3 and RORγ are both required for IL-17 expression, IL-21 expression is regulated by STAT3 but not RORγ.

### Example 6

### The Role of IL-21 in T_{H} Cell Differentiation

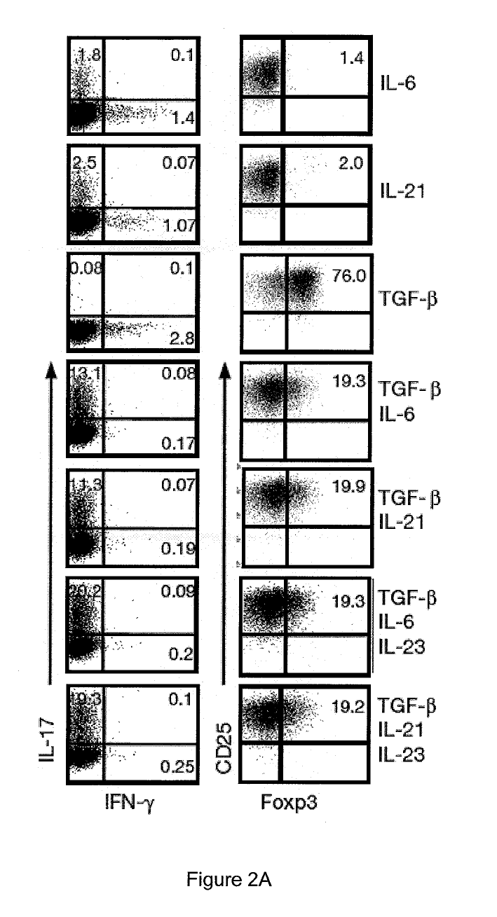

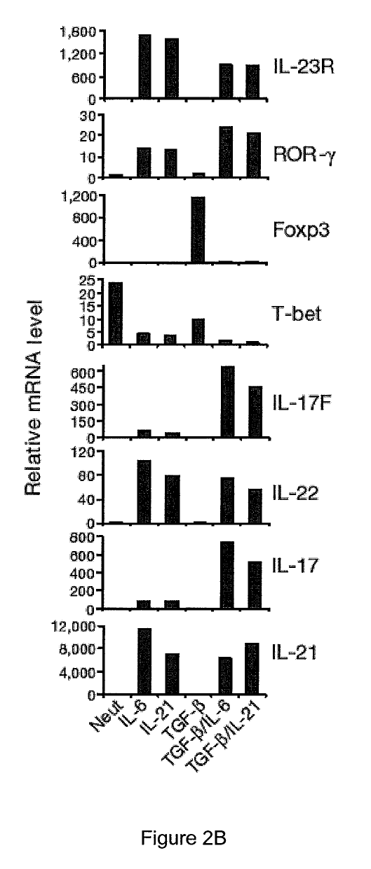

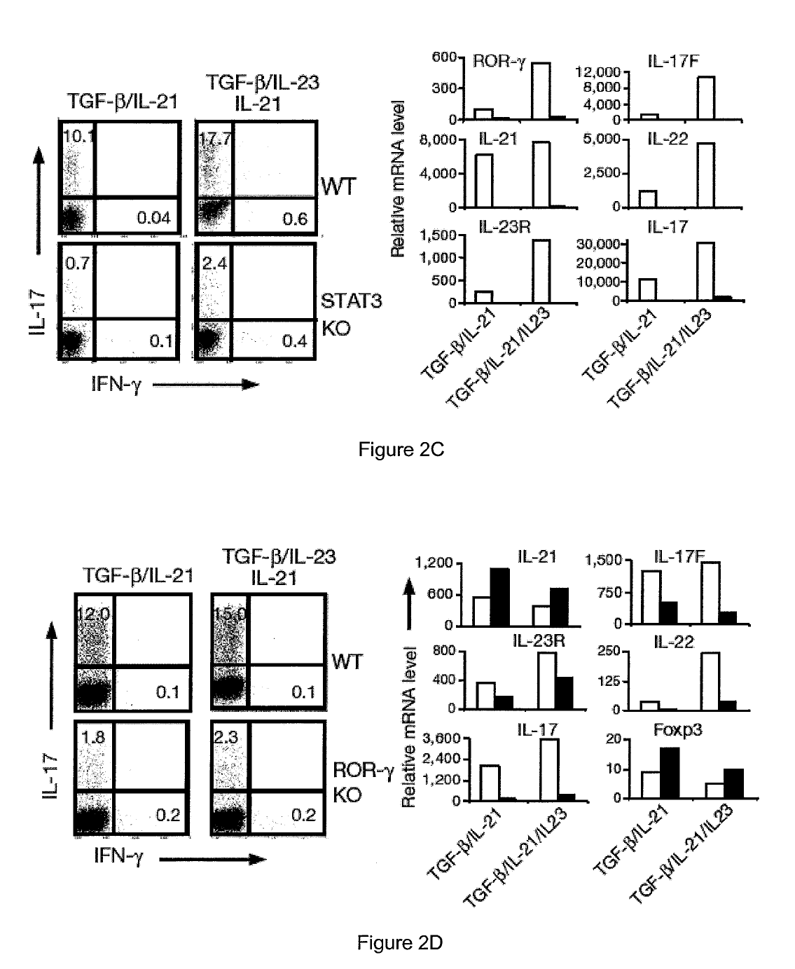

T_{H} cell differentiation is regulated by distinct autocrine cytokines. IFNγ regulates T_{H}1 differentiation, through STAT1 to upregulate T-bet expression while IL-4 regulates T_{H}2 differentiation via STAT6. Afkarian, M., et al., T-Bet Is a STAT1-Induced Regulator of ILL-12R Expression in Naïve CD4⁺ T Cells, 2002, Nature Immunol. 3, 549-557; Glimcher, L. H., et al., Lineage Commitment in the Immune System: The T Helper Lymphocyte Grows Up, 2000, Genes Dev. 14, 1693-1711. To test the possible function of IL-21 in T_{H} differentiation, naïve T_{H} cells from B6 mice were activated in the presence or absence of IL-21. In a similar way to IL-6, IL-21 alone inhibited IFNγ expression but only induced a small percentage of IL-17-producing cells (FIG. 2A). Addition of IL-21 or IL-6 together with TGFβ strongly inhibited Foxp3 expression and greatly enhanced IL-17 production (FIG. 2A). IL-23 synergizes with TGFβ and IL-6 in T_{H}17 differentiation; similarly, it also enhanced the generation of T_{H}17 cells by TGFβ and IL-21 (FIG. 2A). Yang, X. O., et al., STAT3 Regulates Cytokine-Mediated Generation of Inflammatory Helper T Cells, 2007, J. Biol. Chem. 282, 9358-9363.

To further assess the role of IL-21, expression of lineage-specific genes was examined by RT-PCR. IL-21 or IL-6 alone or in combination with TGFβ resulted in upregulation of IL-23R, RORγ and T_{H}17 cytokines IL-17, IL-17F and IL-22, and inhibition of Foxp3 and T-bet mRNA expression (FIG. 2B). These data indicate that IL-21, in a similar way to IL-6, selectively regulates the differentiation of T_{H}17 cells. Moreover, as seen from FIG. 6B, activation of wild-type and Il6^{-/-} T_{H} cells in the presence of TGFβ+IL-21 resulted in similar numbers of T_{H}17 cells (FIG. 7). Thus, IL-21 functions independent of IL-6 in driving T_{H}17 differentiation. Interestingly, no synergistic effect of IL-21 and IL-6 was observed (data not shown), which supports the hierarchy of these two cytokines in T_{H} differentiation.

### Example 7

### Role of STAT3 and RORγ in IL-21-Mediated T_{H}17 Differentiation

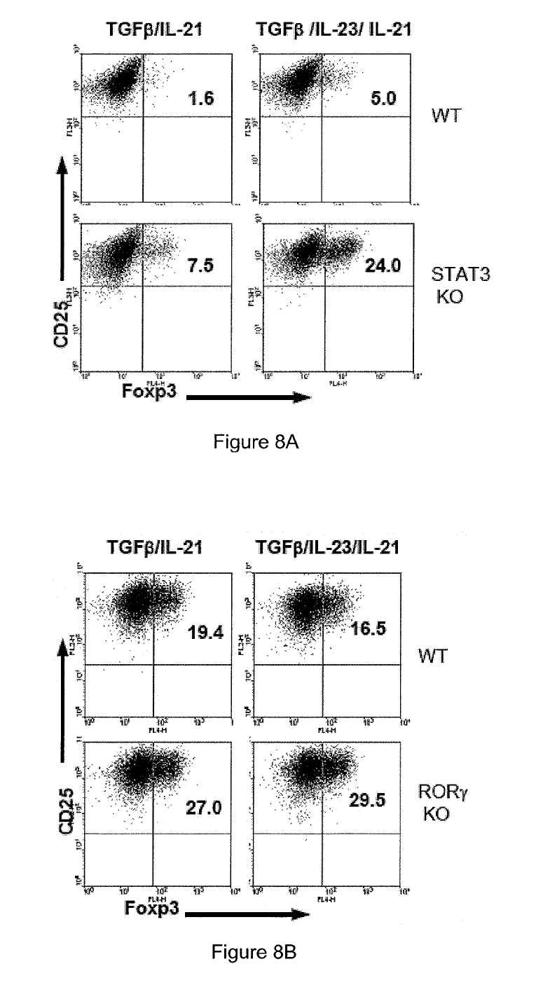

IL-21 activates STAT3 and STAT1 and, to lesser degree, STAT5, among which only STAT3 regulates T_{H}17 differentiation. Leonard, W. J., et al., Interleukin-21: A Modulator of Lymphoid Proliferation, Apoptosis and Differentiation, 2005, Nature Rev. Immunol. 5, 688-698; Yang, X. O., et al., STAT3 Regulates Cytokine-Mediated Generation of Inflammatory Helper T Cells, 2007, J. Biol. Chem. 282, 9358-9363. When naive T cells from STAT3-deficient mice and control mice were differentiated in the presence of IL-21+TGFβ, the expression of IL-17 by STAT3-deficient T cells was greatly reduced and Foxp3 expression was enhanced (FIG. 2C and FIG. 8A). In addition, deficient mRNA expression of IL23R, RORγ, IL-17, IL-17F, IL-22 and IL-21 was observed (FIG. 2C). These data indicate an essential role of STAT3 in IL-21-mediated T_{H}17 differentiation.

Further tests were performed to examine if IL-21-mediated T_{H}17 differentiation is also dependent on RORγ. Naïve T_{H} cells from wild-type and RORγ-deficient mice were differentiated as above. RORγ-deficient T cells were found to produce dramatically reduced levels of IL-17, whereas Foxp3 expression was moderately enhanced (FIGS. 2D and 8B). Real-time PCR analysis also indicated decreased expression of IL23R, IL-17, IL-17F and IL-22 mRNA in RORγ-deficient T_{H} cells compared with the wild-type, while IL-21 production in wild-type and Rorc^{-/-} T_{H} cells remained the same (FIG. 2D). Therefore, IL-21 induces T_{H}17 differentiation and suppress Foxp3 upregulation, in which STAT3 and RORγ are required.

### Example 8

### Role of IL-21 in T_{H}17 Differentiation

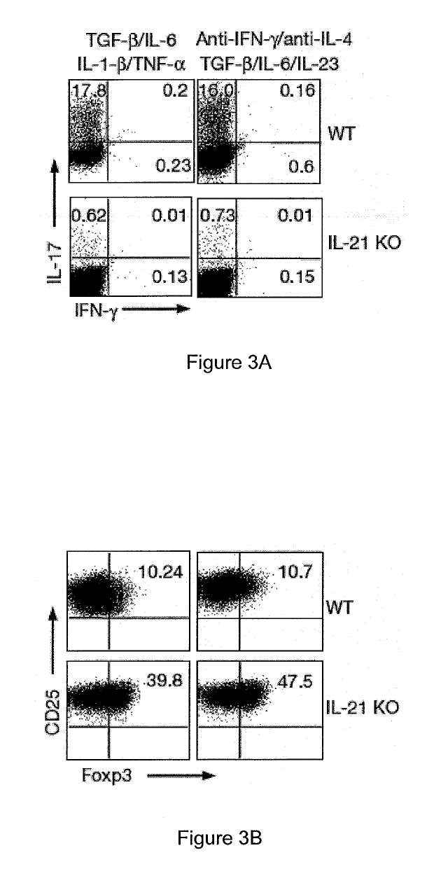

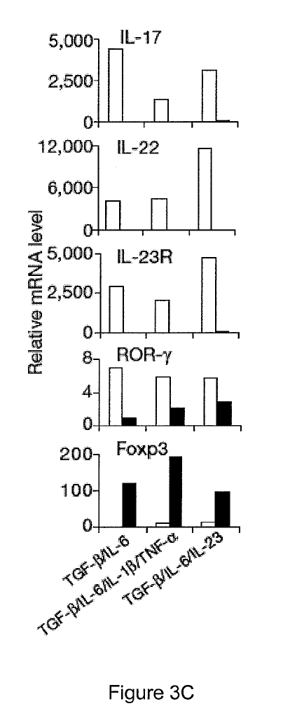

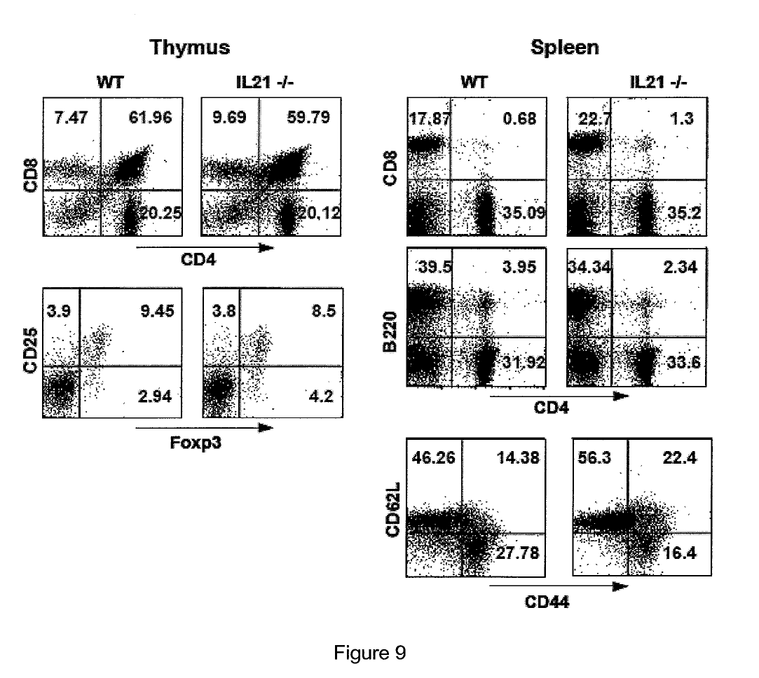

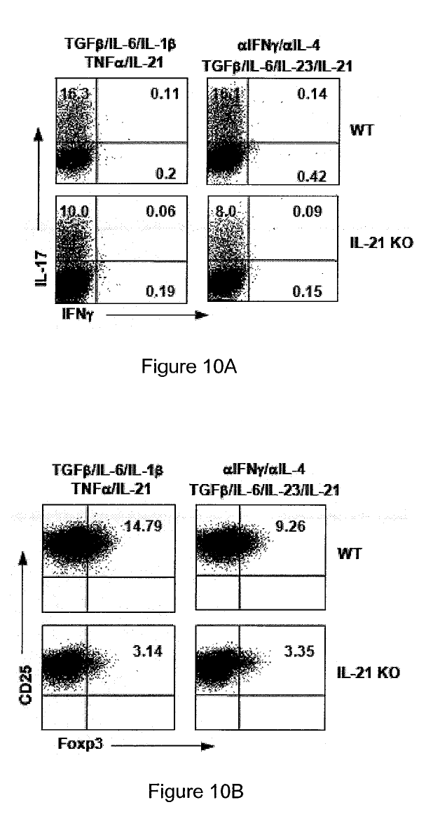

Using an IL-21 knockout mouse, tests were performed to examine if IL-21 is necessary for T_{H}17 differentiation. T_{H}17 cells generated from these mice failed to express IL-21 (data not shown). Similar to IL-21R-deficient mice, IL-21 knockout mice exhibit normal T cell development (FIG. 9). Leonard, W. J., et al., Interleukin-21: A Modulator of Lymphoid Proliferation, Apoptosis and Differentiation, 2005, Nature Rev. Immunol. 5, 688-698; Ozaki, K., et al., A Critical Role for IL-21 in Regulating Immunoglobulin Production, 2002, Science 298, 1630-1634. Naïve wild-type and IL-21−/−T_{H} cells were subject to T_{H}17 differentiation in the presence of IL-6. IL-21-deficient T_{H} cells exhibited deficiency in generation of IL-17-producing cells (FIG. 3A), associated with significantly increased number of Foxp3+ cells compared to wild-type cells (FIG. 3B). Consistently, they were defective in expression of IL23R, IL-17, IL-17F, IL-22 and RORγ mRNA, with increased Foxp3 expression (FIG. 3C). Similarly, IL-21 antagonists also reduced T_{H}17 differentiation (data not shown). Addition of exogenous IL-21 to IL-21-deficient T cells partially restored IL-17 expression and greatly reduced Foxp3 expression (FIGS. 10A and 10B).

### Example 9

### Role of IL-21 in T_{H}17 Differentiation In Vivo; Number of IL-17-Expressing Cells

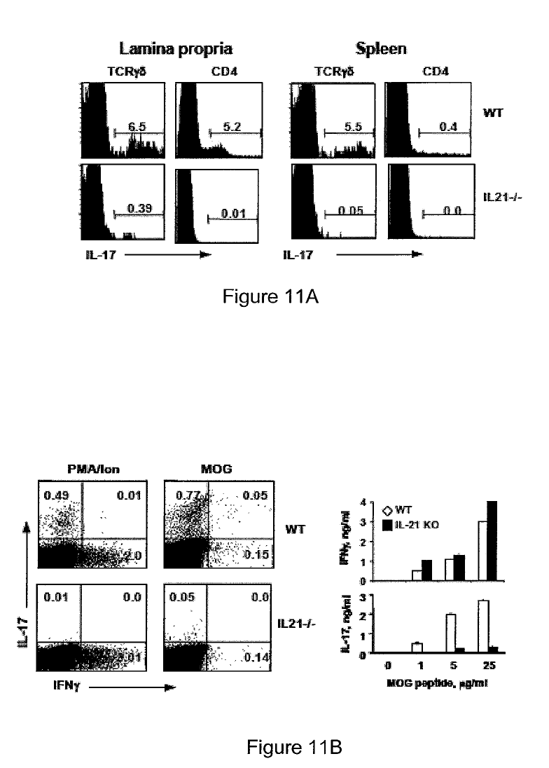

To determine if IL-21 is essential in generation of T_{H}17 cells in vivo, lamina propria and splenic T cells from IL-21-deficient mice were analyzed. In IL-21-deficient mice, lamina propria and splenic TCRγδ+ T cells exhibited at least 10-fold reduction in IL-17-expressing cells compared to those from wild-type mice (FIG. 11A). IL-17+CD4+ cells in lamina propria and spleen were completely absent in Il12^{-/-} mice (FIG. 11A).

### Example 10

### Role of IL-21 in T_{H}17 Differentiation In Vivo; EAE Experiments

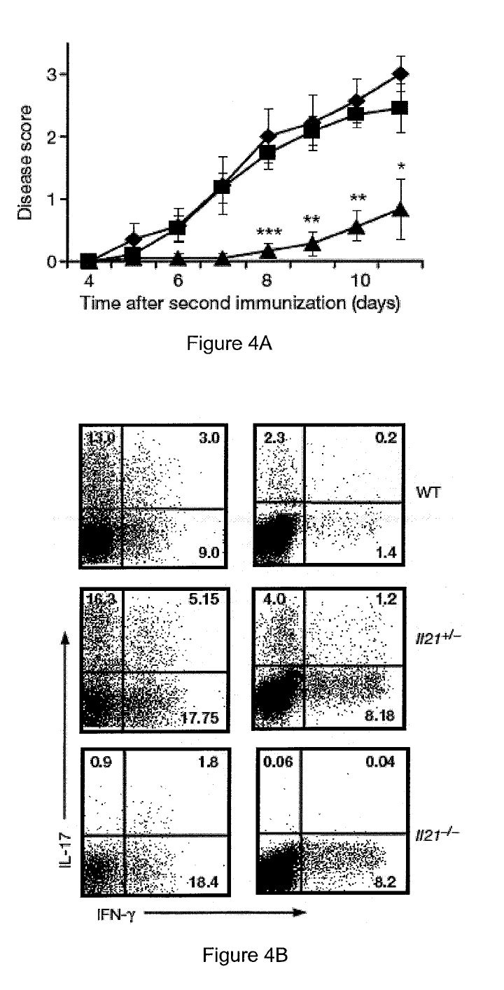

IL-17-producing T_{H} cells have been shown to play an important pathogenic role in experimental autoimmune encephalomyelitis (EAE). Langrish, C. L., et al., IL-23 Drives a Pathogenic T Cell Population That Induces Autoimmune Inflammation, 2005, J. Exp. Med. 201, 233-240; Park, H., et al., A Distinct Lineage of CD4 T Cells Regulates Tissue Inflammation by Producing Interleukin 17, 2005, Nature Immunol. 6, 1133-1141. It has been previously shown that B cells are not important in EAE model. Hjelmstrom, P., et al., B-Cell-Deficient Mice Develop Experimental Allergic Encephalomyelitis With Demyelination After Myelin Oligodendrocyte Glycoprotein Sensitization, 1998, J. Immunol. 161, 4480-4483. IL-21 is important for humoral immunity. Leonard, W. J., et al., Interleukin-21: A Modulator of Lymphoid Proliferation, Apoptosis and Differentiation, 2005, Nature Rev. Immunol. 5, 688-698. On day 5 after second immunization with MOG peptide, wild-type and Il12^{+/-} mice started to develop disease and by day 11 reached score of 2.5-3 (FIG. 4A). In contrast, IL-21-deficient mice first showed signs of disease on day 8 after second immunization; on day 11, only very mild disease were found in these mice (FIG. 4A). Lack of IL-21 thus results in amelioration of EAE.

To understand the underlying cause, the cytokine production by CD4+ cells infiltrating in central nervous system (CNS) and by splenocytes were analyzed further. CNS-infiltrating and splenic CD4+ T cells from wild-type and Il21^{+/-} mice produced similar levels of IL-17; those from Il21^{+/+} mice expressed more IFNγ (FIG. 4B). In contrast, CD4+ cells from IL-21 knockout mice predominately expressed IFNγ, but not IL-17 (FIG. 4B).

To further confirm the role IL-21 in differentiation of IL-17-producing T_{H}17 cells in vivo, wild-type and IL-21-deficient mice were immunized with MOG peptide in CFA. One week later, spleen cells from immunized mice were restimulated with PMA plus ionomycin or MOG peptide ex vivo. Although CD4+ T cells from Il21^{-/-} mice exhibited normal IFNγ expression, IL-17-producing cells was almost completely absent (FIG. 11B). Taken together, these results show that the lack of IL-21 resulted in impaired T_{H}17 differentiation in vivo and in protection against EAE.

### Example 11

### IFNγ and IL-17 Expression in the Presence of Anti-IL-21 Antibodies

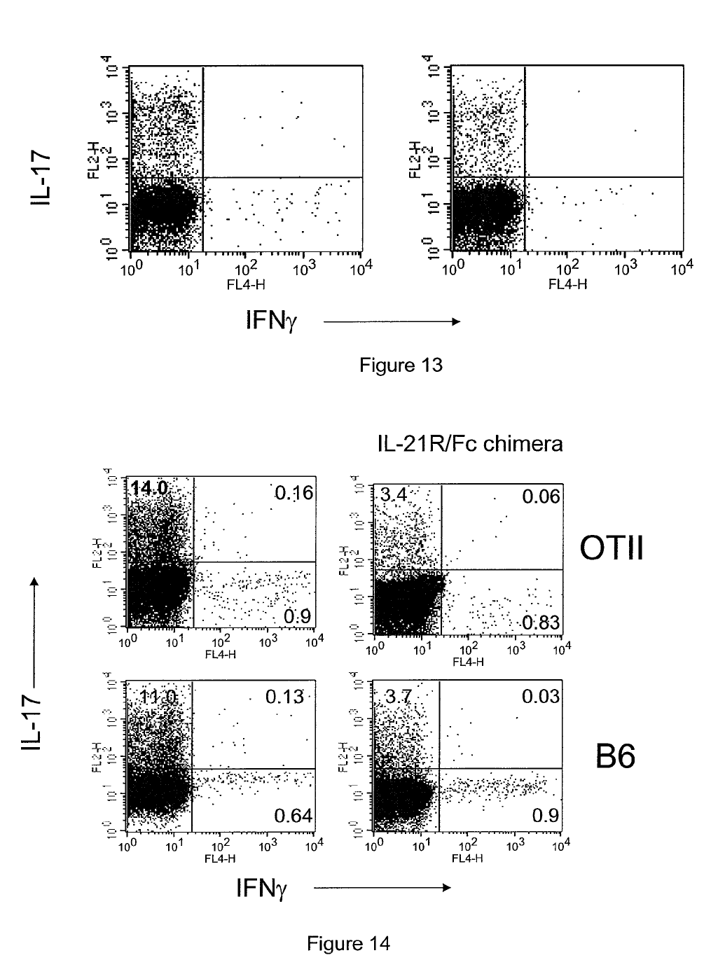

CD4+ T cells from OT-II TcR transgenic mice were activated with Ova peptide and irradiated splenic APC in the present of TGFβ, IL-6, IL-23, anti-IL-4, anti-IFNγ and in the presence or absence of anti mouse IL-21 antibodies. The anti mouse IL-21 antibodies used are commercially available from R&D, catalog number AF594. Five days later, cells were analyzed for IFNγ and IL-17 expression using intracellular staining. The results are shown in FIG. 13.

### Example 12

### IFNγ and IL-17 Expression in the Presence of Soluble IL-21 Receptor Antagonists

CD4+ T cells from OT-II TcR transgenic mice were activated with Ova peptide and irradiated splenic APC in the present of TGFβ, IL-6, and IL-23, anti-IL-4, anti-IFNγ and in the presence or absence of recombinant mouse IL-21 R/Fc chimera. The recombinant mouse IL-21 R/Fc chimera that were used are commercially available from R&D, catalog number 596-MR. FACS-sorted naïve T cells from B6 mice were activated with plate-bound anti-CD3, anti-CD28 and IL-2 in the presence of indicated above cytokines. Five days later, cells were analyzed for IFNγ and IL-17 expression using intracellular staining. The results are shown in FIG. 14.

We claim:

1. A method of treating a disease or condition associated with T_{H}17 cell mediated immune response in a subject in need thereof comprising the step of administering to the subject a therapeutically effective amount of an agent that modulates the IL-21 signaling pathway in an amount effective to modulate the differentiation of T_{H}17 cells or the expression of IL-17, IL17-F, IL-22, and IL-21.

2. A method of treating an inflammatory disease or condition in a subject in need thereof comprising the step of administering to the subject a therapeutically effective amount of an agent that modulates the IL-21 signaling pathway so as to modulate the differentiation of T_{H}17 cells.

3. The method of claim 2 wherein the agent that modulates is an IL-21 antagonist.

4. The method of claim 3 wherein the agent is an anti-IL-21 antibody.

5. A method of modulating the differentiation of T_{H}17 cells comprising the steps of modulating the IL-21 signaling pathway and the expression of IL-17, IL17-F, IL-22, or IL-21 by T_{H}17 cells whereby the generation of T_{H}17 cells is induced or inhibited and an immune response modified.
